(19) United States
(12) Patent Application Publication
(10) Pub. No.: US 2008/0280849 A1
(43) Pub. Date: Nov. 13, 2008

(54) Synergic Combinations Comprising a Quinoline Compound and Other Hiv Infection Therapeutic Agents

(76) Inventors: Herve Leh, Paris (FR); Fatima Zouhiri, Chatenay Malabry (FR); Jean-Francois Mouscadet, Sceaux (FR); Claire-Marie Thomas, Aigrefeuille d' Annie (FR); Elisabeth Dam, Paris (FR); Dominique Costantini, Paris (FR)

Correspondence Address: STITES & HARBISON PLLC, 1199 NORTH FAIRFAX STREET, SUITE 900, ALEXANDRIA, VA, 22314, US

(21) Appl. No.: 11/915,786

(22) PCT Filed: Jun. 1, 2005
(86) PCT No.: PCT/IB2005/001538

§ 371 Date: Nov. 28, 2007

(51) Int. Cl. A61K 31/47; A61K 31/7064; A61K 31/52; A61K 31/708; A61K 31/7068; A61K 31/513; A61K 31/7072; A61K 31/675; A61K 31/496; A61K 31/538; A61K 31/551; A61K 31/472; A61P 31/18; C07D 215/00

(52) U.S. Cl. 514/45; 514/311; 514/49; 514/263.4; 514/274; 514/81; 514/50; 514/253.09; 514/230.5; 514/220; 546/152; 514/307

(57) ABSTRACT

The invention relates to a combination comprising a quinoline compound or its sail, according to general formula (I) and at least one HIV infection therapeutic agent selected from the group consisting of entry inhibitors, reverse-transcriptase inhibitors, strand-transfer inhibitors, protease inhibitors, and maturation inhibitors. Said combination has therapeutic synergy in the treatment of an HIV infection compared with the quinoline compound, or HIV infection therapeutic agent alone.

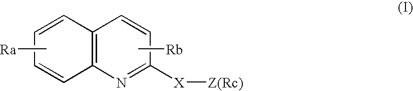

The invention relates to a combination comprising a quinoline compound or its salt, according to general formula (I)

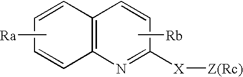

and at least one HIV infection therapeutic agent selected from the group consisting of entry inhibitors, reverse-transcriptase inhibitors, strand-transfer inhibitors, protease inhibitors, and maturation inhibitors. Said combination has therapeutic synergy in the treatment of an HIV infection compared with the quinoline compound or HIV infection therapeutic agent alone.

Antiretroviral therapy for treatment of Human Immunodeficiency Virus type 1 (HIV-1) infection has improved steadily since the advent of combination therapy in 1996. 20 antiretroviral agents have been approved so far, belonging to four classes. These four classes include the nucleoside/nucleotide reverse transcriptase inhibitors (NRTI), non-nucleoside reverse transcriptase inhibitors (NNRTI), protease inhibitors (PI), and fusion inhibitors (FI), with which combination regimens containing at least three drugs have been designed. However the use of reverse transcriptase and protease inhibitors has led to the emergence of resistant strains. This highlights the necessity to develop new targeted drugs such as integrase inhibitors.

Retroviral integration, the process that stably inserts the DNA copy of the viral genomic RNA into the host cell genome, is an essential step for productive infection. After entry of HIV-1 into the cell, the viral capsid seems to uncoat rapidly, and the virion core is released into the cytoplasm of infected cell. The genomic HIV-1 RNA is reverse-transcribed into linear double-stranded DNA and viral components are reorganized into a large nucleoprotein complex, the preintegration complex (PIC), composed of the viral DNA and viral and cellular proteins. Viral DNA is then actively imported into the nucleus through the nuclear envelope of interphase cell. HIV-1 DNA is ultimately integrated into host DNA, which ensures expression and perpetuation of the viral genome. This process is carried out by the viral integrase (IN), which represents therefore a legitimate target for new inhibitors, in that combination therapy with reverse transcriptase and protease inhibitors failed to eradicate viral replication and to prevent emergence of drug-resistant strains.

IN catalyses a two-step process leading to the stable insertion of proviral DNA into the genome of infected cells. In the first step, called 3′ processing, two nucleotides are removed from each 3′-end of the viral DNA. In the second step, the strand transfer reaction, the 3′-processed viral DNA ends are covalently joined to the target DNA.

Although integrase has remained an elusive target for long, several inhibitors displaying antiviral activity have been now identified, including diketo-acids compounds which were shown to specifically inhibit the strand transfer step catalyzed by IN. A new family of inhibitors corresponding to quinoline derivatives has also been recently described (Mekouar et al., 1998; Zouhiri et al., 2000; International patent applications WO 98/45269 and WO 03/031413). Several quinoline derivatives possess antiviral activity in cell culture. In vitro, these compounds are competitive inhibitors of IN binding to DNA that therefore block the 3′-processing activity of the enzyme.

The inventors have demonstrated that a normal level of viral RNA is found in cells infected in the presence of a quinoline derivative whereas the amount of full-length reverse-transcribed DNA is strongly decreased in total cell extracts. Integrase was confirmed to be the probable target because the peculiar mutations (V165I/V249I and C280Y) identified in resistant viruses that have emerged from long term virus culture in the presence of an active quinoline derivative were located within the IN ORF. Furthermore, quinoline derivatives were shown to specifically inhibit nuclear import of IN without affecting other import pathways, whereas specific strand transfer inhibitors did not affect IN import (international patent application WO 03/096965).

The inventors have now shown that although these mutations are sufficient to confer viral resistance toward BA011FZ041, a lead quinoline derivative, they do not alter viral sensitivity toward the previously described diketo acid L-731,988, thus confirming that diketo acids and quinoline derivatives possess different mechanisms of action. Furthermore, quinoline derivatives are herein shown to be fully active against DKAs resistant viruses

Thus, altogether the inventors' results support that, in contrast to strand transfer inhibitors that affect specifically the integration step in infected cells (Hazuda et al., 2000), quinoline derivatives are real integrase inhibitors which act at an early step of the replication cycle, most likely by impairing pre-integration complex formation.

The activity of quinoline compounds against viruses resistant to reverse transcriptase inhibitors (RTIs) was further assessed by the inventors.

It is herein demonstrated that quinoline compounds are fully active against viruses resistant to anti-HIV agents such as strand transfer inhibitors and reverse transcriptase inhibitors. This suggests that HIV multitherapy involving quinoline derivatives could allow circumventing emerging resistances to other antiviral drugs.

Furthermore, the inventors have demonstrated that quinoline derivatives show synergy with antiviral drugs, in particular reverse transcriptase inhibitors and integrase strand transfer inhibitors, thus rendering the use of quinoline derivatives in combination therapy particularly attractive.

### DEFINITION

In the context of the invention, “human Immunodeficiency Virus” (HIV) denotes HIV-1 of any group (A-H or O), or HIV-2 of any group (A, B). Preferably HIV is HIV-1.

A “HIV infection” refers to the condition of a subject or patient which is infected with HIV. HIV infection indifferently denotes an asymptomatic HIV infection or the acquired immunodeficiency syndrome (AIDS), whatever its stage of development.

“Anti-HIV agent” or “HIV infection therapeutic agent” denotes a compound which leads to decreased HIV replication. Known anti-HIV agents either inhibit inverse transcriptase, integrase, protease, or fusion of the virus to the cell membrane.

As used herein, the terms “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

“Patient” or “patient in need thereof” is intended for a human or non-human mammal affected or likely to be affected with HIV. Preferably the patient is a human.

In the context of the invention, the term “treating” or “treatment”, as used herein, means reversing, alleviating, inhibiting the progress of, or preventing the HIV infection, or one or more symptoms of such infection.

The term “therapeutically effective amount” as used herein means that amount of quinoline compound and anti-HIV agents that elicits the biological or medicinal response in a tissue, system, animal or human that is being sought by a researcher, veterinarian, medical doctor or other clinician, which includes alleviation of the symptoms of the HIV infection being treated. For the purpose of prevention, a therapeutically effective amount can alternatively be referred to as a “prophylactic amount” of the active agents.

“Alkyl” means an aliphatic hydrocarbon group which may be straight or branched having about 1 to about 8 carbon atoms in the chain. Branched means that one or lower alkyl groups such as methyl, ethyl or propyl are attached to a linear alkyl chain. “Lower alkyl” means about 1 to about 4 carbon atoms in the chain which may be straight or branched.

“Aryl” means an aromatic monocyclic or multicyclic ring system of about 6 to about 14 carbon atoms, preferably of about 6 to about 10 carbon atoms. The aryl is optionally substituted with one or more “ring system substituents” which may be the same or different, and are as defined herein. Exemplary aryl groups include phenyl or naphthyl, or phenyl substituted or naphthyl substituted.

“Aromatic” means cyclically conjugated aryl or heteroaryl as defined below, which satisfy the Hückel (41+2) Rule and/or with a stability due to delocalization significantly greater than that of a hypothetic localized structure. Preferred aromatic groups include phenyl, halo substituted phenyl and azaheteroaryl.

“Halogen” denotes a Br, Cl, I, F atom.

“BA011FZ041” refers to the quinoline compound 8-hydroxy-2-[2-[(3,4-dihydroxy-5-methoxy-phenyl)ethenyl]]7-quinoline carboxylic acid.

### Integrase Inhibitors

According to their mechanism of action, integrase inhibitors can be classified into two distinct families.

The first one comprises the strand transfer inhibitors (INSTIs) which bind to the IN/Viral DNA substrate complex thereby inhibiting the binding of target DNA. Such compounds impair specifically the second step of integration without altering 3′ processing. Diketo acids (DKAs) and their more recent derivatives, naphthyridine carboxamide, are archetypal members of this class and represent the only family of IN inhibitors that reached clinical development to date (Hazuda et al., 2000; Hazuda et al., 2004). INSTIs may include compounds that also block the 3′ processing step.

The second family consists of integrase binding inhibitors (INBIs). Theses compounds interact with the free enzyme, subsequently impairing the formation of the IN/viral DNA complex. As a consequence, they inhibit 3′ processing as well as strand transfer. Quinoline derivatives are representative of this second class.

Integrase Binding Inhibitors

The present invention concerns the use of a quinoline compound or its salt, according to general formula (I)

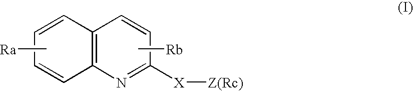

in which Ra, Rb and Rc, identical or different from one another, represent one or more substituents, themselves identical or different, occupying any position on the rings, the substituents being chosen from a —(CH₂)n-Y or —CH—CH—Y group, where Y represents a halogen atom, an —OH, —OR, —COH, —COR, —COOH, —COOR, —COH, —COR, —CONH₂, —CON(Rx,Ry), —CH—NOH, —CO—CH—NOH, —NRxRy, —NO₂, —PO(OR)₂, —SH₂, —SH, —SR, —SO₂R, —SO₂NHR, —CN, —NH(C═O)R, or Z(Rc) radical,

where

R represents an alkyl radical with 1 to 8 carbon atoms, or an aryl or heterocyclic radical,

Rx and Ry, identical or different, represent an hydrogen atom or a linear or branched alkyl radical with 1 to 5 carbon atoms,

Z represents an aryl, heterocyclic radical or an aromatic ring containing heteroatoms chosen from O, N or S, as substitutions for the carbon atoms constituting said aromatic ring, it being possible or otherwise for this ring to be substituted with Rc and

n is zero or an integer from 1 to 5,

Rb moreover can represent a hydrogen atom,

and when Y represents a —COOH or —COOR group in Rc, Z, if it represents an aryl group, includes at least 3 substituents or the quinoline ring is trisubstituted,

X represents an ethylene double bond, or a —(CH₂)n- group, where n is an integer from 1 to 5, or a —(C═O)N(d)X′- group, or a —CH(Rd)—CH(Re)— group, Rd and Re, identical or different, representing a hydrogen atom, halogen atom, a hydroxy or epoxy group, or a —(CH₂)n′-O—C(O)—(CH₂)m-, —(CH₂)n′-C(O)—O—(CH₂)m-, —(CH₂)n′-O—(CH₂)m—, —(CH₂)n′-N(O)—(CH₂)m- or —(CH₂)n′-S(O)t-(CH₂)m- group, where n′ is an integer from 0 to 8,

Rd represents a hydrogen atom or a group —(CH₂)n″-Y′, for which n″ is equal to 0, 1, 2 or 3 and Y′ represents —CH₃, —COOH, —COOR′, —CN, —OH, —OR′, SR′, or an aryl group optionally substituted with Rc, R′ represents a linear or branched alkyl chain of 1 to 4 carbon atoms,

X′ represents an alkyl-(CH₂)n′″- chain in which n′″ is equal to 0, 1 or 2, or O, or N,

m is an integer from 0 to 8, t is zero or an integer equal to 1 or 2, and Q represents a hydrogen atom, an alkyl or aryl radical, as well as the pharmaceutically acceptable salts of these derivatives, their diastereoisomeric forms and their enantiomeric forms.

Preferred compounds of formula (I) are those of formula (Ia):

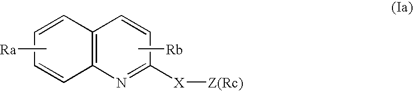

in which Ra, Rb and Rc, identical or different from one another, represent one or more substituents, themselves identical or different, occupying any position on the rings, the substituents being chosen from a —(CH₂)n-Y or —CH—CH—Y group, where Y represents a halogen atom, an —OH, —OR, —COH, —COR, —COOH, —COOR, —COH, —COR, —CONH₂, —CON(Rx,Ry), —CH—NOH, —CO—CH—NOH, —NH₂, —N(Rx,Ry), —NO₂, —PO(OR)₂, —SH₂, —SR, —SO₂R, —SO₂NHR, —NH(C═O)R, —CN, or Z(Rc) radical, where R represents an alkyl radical with 1 to 8 carbon atoms, or an aryl or heterocyclic radical, Rx and Ry, identical or different, represent an alkyl radical with 1 to 5 carbon atoms, Z represents an aryl or heterocyclic radical and n is zero or an integer from 1 to 5, Rb moreover can represent a hydrogen atom, and when Y represents a —COOH or —COOR group in Rc, Z, if it represents an aryl group, includes at least 3 substituents or the quinoline ring is trisubstituted, X represents an ethylene double bond, or a —(CH₂)n- group, where n is an integer from 1 to 5, or a —CH(Rd)—CH(Re)— group, Rd and Re, identical or different, representing a hydrogen atom, halogen atom, a hydroxy or epoxy group, or a —(CH₂)n′-O—C(O)—(CH₂)m-, —(CH₂)n′-C(O)—O—(CH₂)m-, —(CH₂)n′-O—(CH₂)m-, —(CH₂)n′-N(O)—(CH₂)m- or —(CH₂)n′-S(O)t-(CH₂)m- group, where n′ is an integer from 0 to 8, m is an integer from 0 to 8, t is zero or an integer equal to 1 or 2, and Q represents a hydrogen atom, an alkyl or aryl radical,

as well as the pharmaceutically acceptable salts of these derivatives, their diastereoisomeric forms and their enantiomeric forms.

According to another embodiment, preferred compounds of formula (I) are those of formula (Ib):

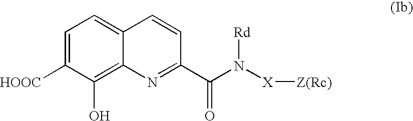

in which

X represents an alkyl-(CH₂)n- chain in which n is equal to 0, 1 or 2, or O or N,

Z represents an aromatic ring which may contain heteroatoms chosen from O, N or S, as substitutions for the carbon atoms constituting said aromatic ring, it being possible or otherwise for this ring to be substituted with Rc,

Rc represents 1 to 3 identical or different substituents chosen from the groups —OH, —OR, —COOH, —COOR, —COH, —COR, —NH₂, —NH(R), —NH(R,R′), —SH, —SR and CN,

Rd represents a hydrogen atom or a group —(CH₂)n″-Y′, for which n″ is equal to 0, 1, 2 or 3 and Y′ represents —CH₃, —COOH, —COOR′, —CN, —OH, —OR′, SR′, or an aryl group optionally substituted with Rc,

R and R′, which are identical or different, represent a linear or branched alkyl chain of 1 to 4 carbon atoms, and

their pharmaceutically acceptable salts, their diastereoisomeric forms and their enantiomeric forms.

In the above formulae (I), (Ia) or (Ib):

Preferably, Ra represents one or more substituents, themselves identical or different chosen from COOH (or a salt thereof), COOR, —CN and/or OH.

Preferably, Ra represents at least one substituent at C-8 position; more preferably 8-OH and/or one substituent at C-7 or C-5 position; more preferably 5-COOH or 7-COOH. Preferably, Ra represents 2 or 3 substituents. Most preferably, Ra represents 7- and/or 5-COOH (or a salt thereof) and 8-OH.

Preferably, Rb represents H.

Preferably, X represents —CH═CH— or —C(═O)—N(Rd)—X′-, wherein Rd, X′ are defined as above. Preferably, Rd represents a hydrogen atom. Preferably, X′ represents —(CH₂)n′″- with n″″ is equal to 0 or 1.

Preferably, Z represents an aryl, more preferably a phenyl group.

Preferably, Rc represent one or more substituents, themselves identical or different chosen from OH, OR, a halogen atom, —NRxRy, —NO₂, —NH(C═O)R, wherein R, Rx and Ry are defined as above. Preferably, R represents an alkyl radical with 1 to 8 carbon atoms; preferably, Rx and Ry, identical or different, represent an hydrogen atom or a linear or branched alkyl radical with 1 to 5 carbon atoms.

More preferably, Rc represents at least two substituents at 3′ and 4′ position of the phenyl group; most preferably, the 4′ substituent is 4′-OH.

Preferably, the pharmaceutically acceptable salt of these derivatives is the sodium salt.

Preferably, compounds of formula (Ia) are chosen from those of formula (Ia′):

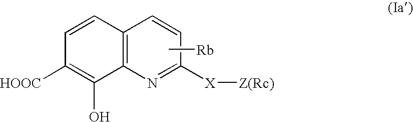

wherein Rb, X, Z, Rc are defined as in formula (Ia).

Most preferred compounds are selected from the group consisting of:
8-hydroxy-2-[2-[(3,4-dihydroxy-5-methoxy-phenyl)ethenyl]]7-quinoline carboxylic acid
8-hydroxy-2-[2-[(3,4-dihydroxy-5-methoxy-phenyl)ethenyl]]5,7-quinoline dicarboxylic acid
8-hydroxy-2-[2-[(3,4-dihydroxy-phenyl)ethenyl]]5,7-quinoline dicarboxylic acid
8-hydroxy-2-[2-[(3-methoxy-4-hydroxy-phenyl)ethenyl]]7-quinoline carboxylic acid
8-hydroxy-2-[2-[(2,3-dihydroxy-phenyl)ethenyl]]7-quinoline carboxylic acid
8-hydroxy-2-[2-[(3,4-dihydroxy-phenyl)ethenyl]]5-quinoline carboxylic acid
8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3,5-dimethoxy-4-hydroxy-phenyl)ethenyl]]7-quinoline carboxylic acid
8-hydroxy-2-[2-[(3-amino-4-hydroxy-5-methoxy-phenyl)ethenyl]]7-quinoline carboxylic acid
8-hydroxy-2-[2-[(3,5-dibromo-4-hydroxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid
2-[2-[(3,4-dihydroxy-phenyl)ethenyl]]-quinoline
8-hydroxy-2-[2-[(3,4-dihydroxy-phenyl)ethenyl]]-quinoline
8-hydroxy-2-[2-[(3,4-dihydroxy-phenyl)ethenyl]]7-quinoline carboxylic acid
7-cyano-8-hydroxy-2-[2-[(3-acetamido-4-hydroxy-5-methoxy-phenyl)ethenyl]]-quinoline
8-hydroxy-2-[2-[(3,4,5-trihydroxy-phenyl)ethenyl]]7-quinoline carboxylic acid
2-[2-[(3,4-dihydroxy-phenyl)ethenyl]]7,8-quinoline dicarboxylic acid
2-(2,3,4-trihydroxy-benzylcarbamoyl)-8-hydroxyquinoline-7-carboxylic acid
2-(2,4-dihydroxy-benzylcarbamoyl)-8-hydroxyquinoline-7-carboxylic acid
2-(3,4-dihydroxy-5-methoxy-phenylcarbamoyl)-8-hydroxyquinoline-7-carboxylic acid
2-(3,4-dihydroxy-benzylcarbamoyl)-8-hydroxyquinoline-7-carboxylic acid
2-(2,3-dihydroxy-benzylcarbamoyl)-8-hydroxyquinoline-7-carboxylic acid
2-(3,4-dihydroxy-phenylcarbamoyl)-8-hydroxyquinoline-7-carboxylic acid

as well as the pharmaceutically acceptable salts of these derivatives, their diastereoisomeric forms and their enantiomeric forms.

According to another object, the present invention also concerns a compound of formula (I) selected from the group consisting of:
8-hydroxy-2-[2-[(3,4-dihydroxy-5-methoxy-phenyl)ethenyl]]5,7-quinoline dicarboxylic acid
8-hydroxy-2-[2-[(2,3-dihydroxy-phenyl)ethenyl]]7-quinoline carboxylic acid
8-hydroxy-2-[2-[(3,4-dihydroxy-phenyl)ethenyl]]5-quinoline carboxylic acid
8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3-amino-4-hydroxy-5-methoxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid

as well as the pharmaceutically acceptable salts of these derivatives, their diastereoisomeric forms and their enantiomeric forms.

Compounds of formula (I), as well as those of formula (Ia), (Ia′) or (Ib) can be prepared by applying or adapting the process of preparation disclosed in WO 98/45269 and WO 03/031413.

The invention further relates to a composition comprising a compound selected from the group consisting in:
8-hydroxy-2-[2-[(3,4-dihydroxy-5-methoxy-phenyl)ethenyl]]5,7-quinoline dicarboxylic acid
8-hydroxy-2-[2-[(2,3-dihydroxy-phenyl)ethenyl]]7-quinoline carboxylic acid
8-hydroxy-2-[2-[(3,4-dihydroxy-phenyl)ethenyl]]5-quinoline carboxylic acid
8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3-amino-4-hydroxy-5-methoxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid

as well as the pharmaceutically acceptable salts of these derivatives, their diastereoisomeric forms and their enantiomeric forms, in a pharmaceutically acceptable carrier.

Preferably said compound is present in the composition in a therapeutically effective amount.

The invention also provides a method of treating a HIV infection comprising administering a patient in need thereof with a composition as defined above.

In the method of treatment provided here, the composition comprising a quinoline compound may have any form known in the art, may be administered in any of route of administration and according to dosage regimens established in the art, as described below in the “Therapeutic methods” section.

Strand Transfer Inhibitors

Other classes of integrase inhibitors have been described in the art. They may be compounds which are selective inhibitors of the strand transfer step mediated by integrase, or compounds which block similarly 3′ processing and strand transfer.

Mention may be made of the strand transfer inhibitors described in the international patent applications WO 99/62513, WO 0230930, WO 02055079, WO 0230426, WO 0230931, WO 0236734, WO 03016315, WO 03062204, WO 03077850, WO 03077857, WO 03086319, WO 2004024078, WO 2004047725, and WO 2004080402 to Merck & Co, Inc., which are incorporated herein by reference.

More specifically, Merck has developed a series of diketo acid compounds, such as L-731,988 (4-[1-(4-fluorobenzyl)pyrrole-2-yl]-2,4-diketobutanoic acid), and L-708,906 (4-(3,5-Bis-benzyloxy-phenyl)-2,4-dioxo-butyric acid), L-731,927 (1H-Pyrrole-2-butanoic acid, a,g-dioxo-1-(3-phenylpropyl)-) as described by Hazuda et al. (2000).

These compounds, which have also been described in the international patent application WO 99/62513, have formula:

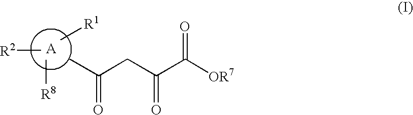

wherein A is a five-membered heteroaromatic ring containing 1 or 2 nitrogen atoms and substituted on carbon or nitrogen by R¹, R², and R⁸; the heteroaromatic ring may optionally be fused with a phenyl ring to form a fused ring system, provided that when A is a fused ring system, the nitrogen-containing heteroaromatic ring is substituted by the dioxobutyric acid/ester moiety;

R¹ is selected from: (1) —H, (2) —C1-5 alkyl, (3) —CF3, (4) -halo, (5) —NO₂, (6) —N(R⁴) (R⁵), (7) -R⁶, (8) —C2-5 alkenyl-R³ (9) —C2-5 alkynyl-R³, (10) —O—R⁶, (11) —O—C1-6 alkyl, and (12) —C(O)CH₂C(O)C(O)OR⁷;

R² is selected from: (1) —H, (2) -R³, (3) —C1-6 alkyl, (4) —C1-6 alkyl substituted with R³, (5) —O—R⁶, (6) —O—C1-6 alkyl-OR⁶, (7) —S(O)n-R⁶, (8) —C1-6 alkyl (OR⁶)(R⁴), (9) —C1-6 alkyl N(R⁴)(R⁶) (10) —C1-6 alkyl S(O)n-R⁶, (11) —C1-6 alkylC(O)—R⁶, (12) —C1-6 alkyl C(S)—R⁶, (13) —C1-6 alkyl NR⁴C(O)—R⁶, and (14) —C1-6 alkyl-C(O)N(R⁴)(R⁵);

each R³ is independently selected from:

(1) a 5 or 6 membered aromatic or heteroaromatic ring, containing 0, 1, 2, 3, or 4 heteroatoms selected from oxygen, nitrogen and sulfur, unsubstituted or substituted on a nitrogen or carbon atom by 1 to 5 substituents selected from: (a) halogen, (b) C1-6 alkyl, (c) C1-6 alkyloxy, (d) phenyl, (e) —CF₃, (f) —OCF₃, (g) —CN, (h) hydroxy, (i) phenyloxy, and (j) substituted phenyloxy with 1, 2, or 3 substituents selected from: (i) halogen, (ii) C1-6 alkyl, (iii) —CF₃, and (iv) hydroxy;

(2) a 3 to 6 membered saturated ring containing 0 or 1 heteroatoms selected from oxygen, nitrogen or sulfur, unsubstituted or substituted with 1 to 5 substituents selected from: (a) halogen, (b) C1-6 alkyl, (c) C1-6 alkyloxy-, (d) —CF₃, (e) —OCF₃, (f) —CN, (g) =0, (h) hydroxy;

(3) unsubstituted or substituted hexahydrothieno[3,4-d]imidazolyl with one or two substituents selected from: (a) oxo, (b) halogen, (c) C1-6 alkyl, (d) alkyloxy-, (e) —CF3, (f) —OCF3, (g) —CN, and (h) hydroxy;

(4) a 5 or 6 membered aromatic or heteroaromatic ring, containing 0, 1, or 2 heteroatoms selected from oxygen, nitrogen and sulfur, fused with a phenyl ring; wherein the ring system is unsubstituted or substituted on a nitrogen or carbon atom by 1 to 3 substituents selected from: (a) -halogen, (b) —C1-6 alkyl, (c) —C1-6 alkyloxy, (d) —CF₃, (e) —OCF₃, (f) —CN, and (g) -hydroxy; (5) a 3 to 6 membered saturated ring containing 0 or 1 heteroatoms selected from oxygen, nitrogen or sulfur, fused with a phenyl ring, unsubstituted or substituted with 1 or 2 substituents selected from: (a) halogen, (b) C1-6 alkyl, (c) C1-6 alkyloxy-, (d) —CF₃, (e) —OCF₃, (f) —CN, (h) hydroxy; and

(6) a 5 to 6 membered ring containing 0, 1 or 2 heteroatoms selected from oxygen, nitrogen or sulfur, containing 2 or 3 double bonds, unsubstituted or substituted with 1 or 2 substituents selected from: (a) halogen, (b) C1-6 alkyl, (c) C1-6 alkyloxy-, (d) —CF3, (e) —OCF3, (f) —CN, (g) ═O, (h) hydroxy;

each R⁴ is independently selected from: (1) —H, (2) —C1-3 alkyl, (3) —CF₃, (5) —C2-3 alkenyl, (6) —C1-3 alkyl-R³, (7) —C2-3 alkenyl-R³, (8) —S(O)n-R³, and (9) —C(O)—R³;

each R⁵ is independently selected from: 1) —H, (2) —C1-3 alkyl, (3) —CF₃, (4) -R³, (5) —C2-3 alkenyl, (6) —C1-3 alkyl-R³, (7) —C2-3 alkenyl-R³, (8) —S(O)n-R and (9) —C(O)—R³;

each R⁶ is independently selected from: (1) —C1-3 alkyl-R³, and (2) -R³;

R⁷ is selected from: (1) —H, and (2) C1-6 alkyl;

R⁸ is selected from: (1) —H, (2) C1-6 alkyl-oxy, and (3) C1-6 alkyl;

and each n is independently selected from 0, 1 and 2;
as well as salt or ester thereof.

Naphthyridine carboxamide derivatives have also been described more recently.

These include in particular the compounds of general formula described in the international patent application WO 03/086319:

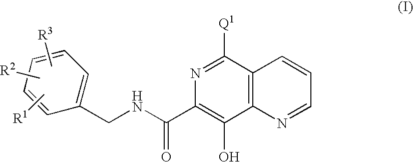

wherein in Formula (I) each of R1, R2 and R3 is independently:

(1) —H, (2) —C1-6 alkyl, optionally substituted with one substituent which is —OH, —O—C1-6 alkyl, —O—C1-6 haloalkyl, —CN, —NO₂, —N(RaRb), —(═O)N(RaRb), —C(═O)Ra, —CO₂RC, —OCO₂RC, —S(O)nRc, -(RaRb), —N(Ra)C(═O)Rb, —N(Ra)CO₂RC, —N(Ra) SO₂RC, —N(Ra) SO₂N(RaRb), —OC(═O)N(RaRb), or —N(Ra)C(═O)N(Ra Rb), (3) —O—C1-6 alkyl, optionally substituted with one substituent which is —OH, —O—C1-6 alkyl, —O—C1-6 haloalkyl, —S(O)nRc, —N(Ra)—CO₂Rc, —C(═O)N(RaRb), —SO₂N(RaRb), —N(Ra)C(═O)Rb, —N(Ra)CO₂Rc, —N(Ra)SO₂Rc, —N(Ra)SO₂N(RaRb), —OC(═O)N (RaRb), or —N(Ra)C(═O)N(RaRb), (4) —C1-6 haloalkyl, (5) —O—C1-6 haloalkyl, (6) —OH, (7) halo, (8) —NO₂, (9) —CN, (10) —C(═O)Ra, (11) —CO₂RC, (12) —S(O)nRc, (13) —SO₂N(RaRb), (14) —N(RaRb), (15) —C(═O)N(RaRb),

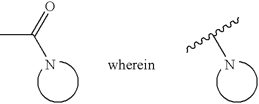

is azetidinyl, pyrrolidinyl, piperidinyl, or morpholino, (17) —N(Ra) SO₂Rc, (18) —OC(═O) N(RaRb), (19) —N(Ra) C(═O)N(RaRb), (20) —N(Ra)—C1-6 alkyl-C(═O)N(RaRb), (21) —N(Ra)—C1-6 alkyl-SRa, (22) —N(Ra)—C1-6 alkyl-ORa, (23) —N(Ra)—C1-6 alkyl-N (Ra)2, (24) —N(Ra)—C1-6 alkylN(Ra)—C(Ra) ═O, (25) —N(Ra)—C(═O)—C1-6 alkyl-N (RaRb), (26) —N(Ra)C(═O)—C(═O)N(RaRb), (27) —OCO₂Rc, (28) —N(Ra)—SO₂N (RaRb), (29) —N(Ra)—SO₂—C1-6 alkyl-N(RaRb), (30) —N(Ra) C(═O)Rb, (31) —N(Ra) CO₂Rc, (32) —S—C1-6 alkyl-C(═O)N(RaRb), (33) —N(SO2Rc)-C1-6 alkyl-C(═O)N (RaRb), (34) —N(Ra)—C(═O)—C1-6 alkyl-C(═O)N(RaRb), (35) —N(Ra)—C(═O)—C1-6 alkyl-N(Ra)C(═O) (Rb), (36) —N(Ra)—SO₂C1-6 alkyl-C(═O)N(RaRb), (37) —N(Ra)—SO₂—C1-6 alkyl-N(Ra)C(═O) (Rb), (38) —C(═O)N(Ra)—C1-6 alkyl-C(═O)N(RaRb), (39) —C(═O)N(Ra)—C1-6 alkyl-N(Ra) C(═O) (Rb), with the proviso that the —N(Ra)— moieties are not both attached to the same carbon atom of the —C1-6 alkyl-moiety, (40) —C(═O)N(Ra)—C1-6 alkyl-O—C1-3 alkyl, with the proviso that the —N(Ra)— moiety and the —O—C1-3 alkyl group are not both attached to the same carbon atom of the —C1-6 alkyl-moiety, or (41) —C(═O)N(Ra)—C1-6 alkyl-S(O)nRc;

Q¹ is: (1) —H, (2) —C(═O)N(RaRb), (3) —C1-6 alkyl-C(═O)N(RaRb), (4) —S—C1-6 alkyl-C(═O)N(RaRb), (5) —O—C1-6 alkyl-C(═O)N(RaRb), (6) —N(Ra)—C(Rb)═O (7) —N(SO₂Rc)—C1-6 alkyl-C(═O)N(RaRb), (8) —N(Ra)—C(═O)—C(═O)—N(RaRb), (9) —N (Ra) SO₂Rc, (10) —SO₂N(RaRb), (11) —CH═CH—C(═O)—N(RaRb), (12) —N(Ra)—C1-6 alkyl-C(═O)N(RaRb), (13) —N(Ra)—C(═O)—N(RaRb), (14) -HetC, (15) —C1-C6 alkyl-HetC, or (16) —N(Ra)—C1-6 alkyl-HetC;

HetC is a 5- to 7-membered saturated heterocyclic ring containing from 1 to 4 heteratoms independently selected from N, O and S, wherein the saturated heterocyclic ring is optionally substituted with from 1 to 4 substituents each of which is independently halogen, —C1-4 alkyl, —C3-6 cycloalkyl, —O—C1-4 alkyl, —C14 haloalkyl, —O—C1-4 haloalkyl, —CN, oxo, phenyl, benzyl, phenylethyl, —(CH2)₀₋₃C(═O)N(RaRb), —(CH2)₀₋₃C(═O)Ra, —N(Ra)—C(═O)Rb, N(Ra)—CO₂Rc, —(CH2)₁₋₃N (Ra)—C(═O)Rb, —N(RaRb), —(CH2)₁₋₃N(RaRb), —SO₂Rc, —(CH2)₀₋₃C(═O)-HetD, -HetD, —N(Ra)-HetD, and —(CH2)₁₋₃-HetD; wherein each HetD is independently a 5- or 6-membered heteroaromatic ring containing from 1 to 4 nitrogen atoms or a 5- or 6-membered saturated heterocyclic ring containing from 1 to 4 nitrogen atoms, wherein the ring is optionally substituted with 1 or 2 substituents each of which is independently halogen, oxo, —C1-4 alkyl, or —O—C1-4 alkyl;

each Ra is independently-H, —C1-6 alkyl, —C1-6 haloalkyl, or —C3-6 cycloalkyl;

each Rb is independently-H, —C1-6 alkyl, —C1-6 haloalkyl, or —C3-6 cycloalkyl;

each Rc is independently-C1-6 alkyl, —C1-6 haloalkyl, or —C3-6 cycloalkyl; and

each n is independently an integer equal to zero, 1, or 2.

Preferred naphthyridine carboxamide derivatives include L-870,810 (5-(1,1-dioxido-1,2-thiazinan-2-yl)-N-(4-fluorobenzyl)-8-hydroxy-1,6-naphthyridine-7-carboxamide), L-870,812 (8-hydroxy-5-N-methyl-N′-(2-dimethylamino-1,2-diketo)ethylamino-1,6-naphthyridine-7-(4′-fluorobenzyl)-carboxamide) or a sodium salt thereof (international patent application WO 03/016315).

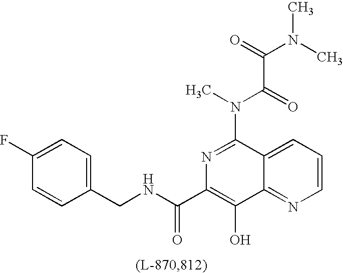

Other strand transfer inhibitors have been described. For instance heteroaromatic derivatives have been described in the U.S. Pat. No. 6,645,956, which is incorporated herein by reference. These have the general formula:

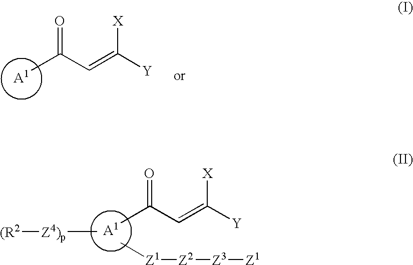

wherein X is hydroxy, protected hydroxy or optionally substituted amino;

Y is —COOR^{A} wherein R^{A} is hydrogen or ester residue, —CONR^{B}R^{c} wherein R^{B} and R^{C} each is independently hydrogen or amide residue, optionally substituted aryl or optionally substituted heteroaryl;

A¹ is optionally substituted heteroaryl;

Z¹ and Z³ each is independently a bond, lower alkylene or lower alkenylene;

Z² and Z⁴ each is independently a bond, lower alkylene, lower alkenylene, —CH(OH)—, —S—, —SO—, —SO₂—, —SO₂NR²¹—, —NR²¹SO₂—, —O—, —NR²¹—, —NR²¹CO—, —CONR²¹—, C(═O)—O—, —O—C(═O)— or —CO—; R²¹ is hydrogen, lower alkyl or lower alkenyl;

R¹ is optionally substituted aryl, optionally substituted heteroaryl, optionally substituted cycloalkyl, optionally substituted cycloalkenyl or optionally substituted heterocycle;

R² is optionally substituted lower alkyl, optionally substituted lower alkyloxy, optionally substituted lower alkyloxycarbonyl, optionally substituted aryl, optionally substituted aryloxy, optionally substituted aryloxycarbonyl, carboxy, optionally substituted cycloalkyl, hydroxy, mercapto, optionally substituted amino, nitro or halogen;

and p is 0 or 1.

Preferably the heteroaromatic derivative is S-1360 (GW810781; 1-[5-(4-fluorobenzyl)furan-2-yl]-3-hydroxy-3-(1H-1,2,4-triazol-3-yl)-propenone), which is under development by Shionogi and GlaxoSmithKline, and along in clinical trials.

The European patent application EP 1375486, which is incorporated herein by reference, discloses further HIV integrase inhibitors which are nitrogen containing heteroaryl compounds of formula (I):

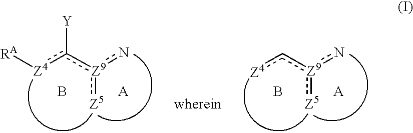

is a condensed nitrogen-containing heterocycle (A ring is nitrogen-containing heterocycle; B ring is carbon ring or heterocycle; Z⁴, Z⁵ and Z⁹ each is independently carbon atom or nitrogen atom);

Y is hydroxy, mercapto or amino;

R^{A} is a group of the formula:

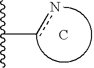

(wherein C ring is nitrogen-containing heteroaryl)

or a group of the formula:

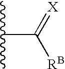

(wherein X is oxygen atom, sulfur atom or NH; R^{B} is hydrogen or a group selected from the substitution group A);
and at least one of A ring, B ring and R^{A} is substituted with a group of the formula: -Z¹-Z²-Z³-R¹ (wherein Z¹ and Z³ each is independently a bond, optionally substituted alkylene or optionally substituted alkenylene; Z² is a bond, optionally substituted alkylene, optionally substituted alkenylene, —CH(OH)—, —S—, —SO—, —SO₂—, —SO₂NR²—, —NR²SO₂—, —O—, —NR²—, —NR²CO—, —CONR²—, —C(═O)—O—, —O—C(═O)— or —CO—; R² is hydrogen, optionally substituted alkyl, optionally substituted alkenyl, optionally substituted aryl or optionally substituted heteroaryl; R¹ is optionally substituted aryl, optionally substituted heteroaryl, optionally substituted cycloalkyl, optionally substituted cycloalkenyl or optionally substituted heterocycle.);
and A ring, B ring or R^{A} is optionally substituted with one to six group(s) selected from the substitution group A at any position except for the position, at which the group shown by the above-mentioned formula: -Z¹-Z²-Z³-R¹ (wherein Z¹, Z², Z³ and R¹ are the same meanings as above.) is substituted, each broken line shows the presence or absence of a bond, except that each neighbouring broken line simultaneously shows the presence of a bond, provided a compound of the formula:

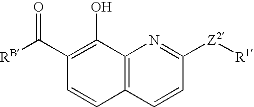

wherein RB′ is hydroxy or alkoxy, Z2′ is alkylene or alkenylene, R1′ is optionally substituted aryl or optionally substituted heteroaryl;

5-benzyl-7-acetyl-8-hydroxyquinoline and 5-phenyl-7-acetyl-8-hydroxyquinoline are excluded.

Integrase inhibitors have been reviewed by Di Santo et al. (2003) which is incorporated herein by reference. Examples of additional integrase inhibitors described therein include cyclohexanone derivatives such as RDS 1028, RDS 1158, RDS 1190, RDS 1211, RDS 1222, RDS 1195; trihydroxycinnamoyl derivatives such as RDS 1468, RDS 1455, RDS 1321, and RDS 1351; carboxylic derivatives such as RDS 1473, RDS 1541, RDS 1572; or aryldiketohexenoic acids such as 5CITEP (1-(5-chloroindol-3-yl)-3-hydroxy-3-(2H-tetrazol-5-yl)-propenone).

### Reverse Transcriptase inhibitors

Reverse transcriptase inhibitors include nucleoside and nonnucleoside reverse transcriptase inhibitors. These may be approved molecules or molecules under development.

Nucleoside Reverse Transcriptase Inhibitors (NRTIs)

NRTIs contain “faulty” versions of nucleotides used by reverse transcriptase to convert RNA to DNA. When reverse-transcriptase uses NRTIs, the viral DNA cannot be built correctly. Thus HIV DNA cannot be incorporated into cellular genome and production of new viruses is prevented.

Any nucleoside reverse transcriptase inhibitor may be used in the combination and method according to the invention.

Preferably, a nucleoside reverse transcriptase inhibitor may be selected in the group consisting of NRTIs which have been approved by the FDA so far. These compounds are reported in the Table 1 below.

TABLE 1
approved nucleoside reverse transcriptase inhibitors

| Brand Name | Generic Name | Manufacturer Name |
|---|---|---|
| Combivir | lamivudine and zidovudine | GlaxoSmithKline |
| Emtriva | FTC, emtricitabine | Gilead Sciences |
| Epivir | lamivudine, 3TC | GlaxoSmithKline |
| Epzicom | abacavir/lamivudine | GlaxoSmithKline |
| Hivid | zalcitabine, ddC, dideoxycytidine | Hoffmann-La Roche |
| Retrovir | zidovudine, AZT, azidothymidine, | GlaxoSmithKline |
| | ZDV | |
| Trizivir | abacavir, zidovudine, and | GlaxoSmithKline |
| | lamivudine | |
| Truvada | tenofovir disoproxil/emtricitabine | Gilead Sciences, Inc. |
| Videx EC | enteric coated didanosine | Bristol Myers-Squibb |
| Videx | didanosine, ddl (dideoxyinosine) | Bristol Myers-Squibb |
| (generic | Didanosine (ddl) | Barr Laboratories, Inc. |
| version) | Delayed Release capsules | |
| Viread | tenofovir disoproxil fumarate | Gilead |
| Zerit | stavudine, d4T | Bristol Myers-Squibb |
| Ziagen | Abacavir | GlaxoSmithKline |

Accordingly, a nucleoside reverse transcriptase inhibitor may be selected from the group consisting of 3TC, AZT (3′-azido-3′-deoxythymidine), azidothymidine, abacavir, d4T, didanosine, 2′,3′-dideoxyinosine (ddI), 2′,3′-dideoxycytidine (ddC), emtricitabine, FTC, lamivudine, stavudine, tenofovir disoproxil/emtricitabine, tenofovir disoproxil fumarate, zalcitabine, and zidovudine (ZDV). Preferably said NRTI is, zidovudine, lamivudine, ddI, or stavudine.

Furthermore, a nucleoside reverse transcriptase inhibitor may a drug under experimentation which has not been approved yet by regulation authorities. Accordingly, the NRTI may further be selected from the group consisting of alovudine (3′-Fluoro-3′-deoxythymidine, also known as MIV-310, co-developed by Medivir and Boehringer Ingelheim), amdoxovir (2R-cis-4-(2,6-diamino-9H-purin-9-yl)-1,3-dioxolane-2-methanol, DAPD) (identified by Triangle Pharmaceuticals, Emory University, and the University of Georgia), and elvucitabine (2,3-Dideoxy-2,3-didehydro-beta-L-fluorocytidine also known as ACH-126,443 or Beta-L-Fd4C, is an L-cytosine nucleoside analog developed by Achillion Pharmaceuticals).

Nonnucleoside Reverse Transcriptase Inhibitors (NNRTIs)

NNRTIs attach themselves to reverse transcriptase and prevent the enzyme from converting RNA to DNA. Thus HIV genetic material cannot be incorporated into cellular genome, and new viruses cannot be produced.

Any nonnucleoside reverse transcriptase inhibitor may be used in the combination and method according to the invention.

Preferably, a nonnucleoside reverse transcriptase inhibitor may be selected in the group consisting of NNRTIs which have been approved by the FDA so far. These compounds are reported in the Table 2 below.

TABLE 2
approved nonnucleoside reverse transcriptase inhibitors

| Brand Name | Generic Name | Manufacturer Name |
|---|---|---|
| Rescriptor | delavirdine, DLV | Pfizer |
| Sustiva | Efavirenz | Bristol Myers-Squibb |
| Viramune | nevirapine, BI-RG-587 | Boehringer Ingelheim |

Accordingly, a nonnucleoside reverse transcriptase inhibitor may be selected from the group consisting of delavirdine, efavirenz, and nevirapine. Preferably said NNRTI is nevirapine or efavirenz.

Furthermore, a nucleoside reverse transcriptase inhibitor may a drug under experimentation which has not been approved yet by regulation authorities. Accordingly, the NNRTI may further be selected from the group consisting of calanolide A (2H,6H,10H-Benzo(1,2-b:3,4-b′:5,6-b″)tripyran-2-one, 11,12-dihydro-12-hydroxy-6,6,10,11-tetramethyl-4-propyl-, (10R-(10alpha,11 beta,12alpha)) developed by Sarawak MediChem Pharmaceuticals), capravirine (5-(3,5-dichlorophenyl)thio-4-isopropyl-1-(4-pyridyl)methyl-1H-imidazol-2-ylmethyl carbamate, CPV) (also known as AG-1549 or S-1153, developed by Agouron Pharmaceuticals/Pfizer), etravirine (4-[[6-amino-5-bromo-2-[(4-cyanophenyl)amino]-4-pyrimidinyl]oxy]-3,5-dimethylbenzonitrile, also known as TMC-125, developed by Tibotec), TMC-120 (4-({4-[(2,4,6-Trimethylphenyl)amino]pyrimidin-2-yl}amino)benzenecarbonitrile) and TMC-278 ((E) 4-[[4-[[4-(2-cyanoethenyl)-2,6-dimethylphenyl]amino]-2-pyrimidinyl]amino]benzonitrile, which is developed by Tibotec), and BMS-561390 (2(1H)-Quinazolinone, 6-chloro-4-[(1E)-2-cyclopropylethenyl]-3,4-dihydro-4-(trifluoromethyl)-, (4S), or DPC-083) (developed by Bristol-Myers Squibb).

### Protease Inhibitors (PIs)

Protease inhibitors prevent T-cells that have been infected with HIV from producing new copies of the virus by blocking processing of HIV polyprotein into structural and non structural proteins.

Any protease inhibitor either approved or under development, may be used in the combination and method according to the invention.

Preferably, a protease inhibitor may be selected in the group consisting of P is which have been approved by the FDA so far. These compounds are reported in the Table 3 below.

TABLE 3
approved protease inhibitors

| Brand Name | Generic Name | Manufacturer Name |
|---|---|---|
| Agenerase | Amprenavir | GlaxoSmithKline |
| Crixivan | indinavir, IDV, MK-639 | Merck |
| Invirase | saquinavir mesylate, SQV | Hoffmann-La Roche |
| Kaletra | lopinavir and ritonavir | Abbott Laboratories |
| Lexiva | Fosamprenavir Calcium | GlaxoSmithKline |
| Norvir | ritonavir, ABT-538 | Abbott Laboratories |
| Reyataz | atazanavir sulfate | Bristol-Myers Squibb |
| Viracept | Nelfinavir mesylate, NFV | Agouron Pharmaceuticals |

Accordingly, a protease inhibitor may be selected from the group consisting of amprenavir, atazanavir, fosamprenavir, indinavir, lopinavir, mesylate, nelfinavir, ritonavir, and saquinavir. Preferably said protease inhibitor is indinavir or saquinavir.

Furthermore, a protease inhibitor may a drug under experimentation which has not been approved yet by regulation authorities, e.g. aptivus (tipranavir, 2-Pyridinesulfonamide, N-[3-[(1R)-1-[(6R)-5,6-dihydro-4-hydroxy-2-oxo-6-(2-phenylethyl)-6-propyl-2H-pyran-3-yl]propyl]phenyl]-5-(trifluoromethyl)-) (developed by Boehringer Ingelheim), or TMC-114 (CAS Number: 618109-00-5, developed by Tibotec).

### Entry Inhibitors (Including Fusion Inhibitors)

Entry inhibitors work by attaching themselves to proteins on the surface of T-cells or proteins on the surface of HIV. In order for HIV to bind to T-cells, the proteins on HIV's outer coat must bind to the proteins on the surface of T-cells. Entry inhibitors prevent this from happening. Some entry inhibitors target the gp120 or gp41 proteins on HIV's surface. Some entry inhibitors target the CD4 protein or the CCR5 or CXCR4 receptors on a T-cell's surface.

Any entry inhibitor may be used in the combination and method according to the invention.

Preferably, a fusion inhibitor may be the fusion inhibitor which has been approved by the FDA, i.e. enfuvirtide (Fuzeon®, Hoffmann-La Roche & Trimeris).

Additionally, the entry inhibitor may a drugs under development, e.g. drugs targeting proteins on T-cells: PRO-542 (a tetravalent CD4-immunoglobulin (Ig) fusion protein that comprises the D1 and D2 domains of human CD4 genetically fused to the heavy and light chain constant regions of human IgG2 (CAS Registry Number 383198-58-1) developed by Progenics Pharmaceuticals) and TNX-355 (monoclonal antibody developed by Tanox and Biogen Idec) target the CD4 protein, and SCH-417,690 (1-(4,6-Dimethyl-pyrimidin-5-yl)-1-(4-{(S)-4-[(R)-2-methoxy-1-(4-trifluoromethyl-phenyl)-ethyl]-3-methyl-piperazin-1-yl}-4-methyl-piperidin-1-yl)-methanone, developed by Schering-Plough Corporation), GSK-873,140 (Benzoic acid,4-(4-(((3R)-1-butyl-3-((R)-cyclohexylhydroxymethyl)-2,5-dioxo-1,4,9-triazaspiro(55)undec-9-yl)methyl)phenoxy), developed by GlaxoSmithKline) and maraviroc (Cyclohexanecarboxamide, 4,4-difluoro-N-((1S)-3-((3-exo)-3-(3-methyl-5-(1-methylethyl)-4H-1,2,4-triazol-4-yl)-8-azabicyclo(3.2.1)oct-8-yl)-1-phenylpropyl)-, CAS number 376348-65-1) (developed by Pfizer) target the CCR5 protein. The entry inhibitor may also be TAK652 ((S)-8-[4-(2-butoxyethoxy)phenyl]-1-isobutyl-N-(4-{([1-propyl-1H-imidazol-5-yl)methyl]sulfinyl}phenyl)-1,2,3,4-tetrahydro-1-benzazocine-5-carboxamide monomethanesulfonate) which is developed by Takeda.

### Maturation Inhibitors

Virus maturation is the process that occurs during the last stages of HIV reproduction, after the virus has been released from the infected cell. It involves the processing of viral proteins and is required for the virus to become infectious. By blocking, or inhibiting, the virus maturation process, new virus cannot go on to infect other cells in the body.

Any maturation inhibitor may be used in the combination and method according to the invention, e.g. PA457 (3-O-(3′,3′-dimethylsuccinyl) betulinic acid), an experimental compound developed by Panacos Pharmaceuticals.

### Combinations

The synergism of quinoline derivatives with other antiretroviral agents, which has been demonstrated by the inventors, makes it possible to provide improved HIV infection therapy using the quinoline derivatives in Highly Active Anti Retroviral Therapy (HAART). HAART is the term used by the skilled in the art to designate a combination treatment of multiple anti-HIV drugs, or multitherapy.

The invention thus relates to a combination comprising an integrase binding inhibitor as defined above, in combination with at least one anti-HIV agent selected from the group consisting of an entry inhibitor (in particular a fusion inhibitor), an reverse-transcriptase inhibitor, a strand-transfer inhibitor, a protease inhibitor, and a maturation inhibitor.

According to an embodiment the quinoline compound as defined above may be used in HAART, i.e. in combination with one or more inhibitor belonging to another class of antiretroviral agents.

The combination may comprise a quinoline compound in combination with at least a strand transfer inhibitor.

The combination may comprise a quinoline compound in combination with a reverse transcriptase inhibitor. In particular said combination may comprise a nucleoside reverse transcriptase inhibitor and/or a nonnucleoside reverse transcriptase inhibitor.

The combination may comprise a quinoline compound in combination with at least a protease inhibitor.

The combination may comprise a quinoline compound in combination with at least an entry inhibitor, in particular a fusion inhibitor.

The combination may comprise a quinoline compound in combination with at least a maturation inhibitor.

According to another embodiment, the quinoline compound as defined above may be used in HAART, i.e. in combination with inhibitors belonging to two other classes of antiretroviral agents.

Thus, the combination may comprise a quinoline compound in combination with a nucleoside reverse transcriptase inhibitor and a nonnucleoside reverse transcriptase inhibitor.

The combination may also comprise a quinoline compound in combination with a strand transfer inhibitor, and a reverse transcriptase inhibitor. In particular said combination may comprise a nucleoside reverse transcriptase inhibitor and/or a nonnucleoside reverse transcriptase inhibitor.

The combination may also comprise a quinoline compound in combination with a strand transfer inhibitor, and a protease inhibitor.

The combination may also comprise a quinoline compound in combination with a strand transfer inhibitor, and an entry (in particular fusion) inhibitor.

The combination may also comprise a quinoline compound in combination with a strand transfer inhibitor, and a maturation inhibitor.

Additionally, the combination may comprise a quinoline compound in combination with a reverse transcriptase inhibitor, and a protease inhibitor. In particular said combination may comprise a nucleoside reverse transcriptase inhibitor and/or a nonnucleoside reverse transcriptase inhibitor.

The combination may also comprise a quinoline compound in combination with a reverse transcriptase inhibitor, and an entry (in particular fusion) inhibitor. In particular said combination may comprise a nucleoside reverse transcriptase inhibitor or a nonnucleoside reverse transcriptase inhibitor.

The combination may also comprise a quinoline compound in combination with a reverse transcriptase inhibitor, and a maturation inhibitor. In particular said combination may comprise a nucleoside reverse transcriptase inhibitor and/or a nonnucleoside reverse transcriptase inhibitor.

The combination may also comprise a quinoline compound in combination with a protease inhibitor, and an entry (in particular fusion) inhibitor.

The combination may also comprise a quinoline compound in combination with a protease inhibitor, and a maturation inhibitor.

The combination may also comprise a quinoline compound in combination with an entry (in particular fusion) inhibitor and a maturation inhibitor.

According to still another embodiment, the quinoline derivative as defined above is used in HAART, i.e. in combination with inhibitors belonging to three other classes of antiretroviral agents.

Accordingly, the combination may comprise a quinoline compound in combination with a strand transfer inhibitor, a reverse transcriptase inhibitor, and a protease inhibitor.

The combination may comprise a quinoline compound in combination with a strand transfer inhibitor, a reverse transcriptase inhibitor, and an entry (in particular a fusion) inhibitor.

The combination may comprise a quinoline compound in combination with a strand transfer inhibitor, a reverse transcriptase inhibitor, and a maturation inhibitor.

The combination may comprise a quinoline compound in combination with a strand transfer inhibitor, a protease inhibitor, and an entry (in particular a fusion) inhibitor.

The combination may comprise a quinoline compound in combination with a strand transfer inhibitor, a protease inhibitor, and a maturation inhibitor.

The combination may comprise a quinoline compound in combination with a strand transfer inhibitor, an entry (in particular a fusion) inhibitor, and a maturation inhibitor.

The combination may comprise a quinoline compound in combination with a reverse transcriptase inhibitor, a protease inhibitor, and an entry (in particular a fusion) inhibitor.

The combination may comprise a quinoline compound in combination with a reverse transcriptase inhibitor, a protease inhibitor, and a maturation inhibitor.

The combination may comprise a quinoline compound in combination with a reverse transcriptase inhibitor, an entry (in particular a fusion) inhibitor, and a maturation inhibitor.

According to this embodiment, the reverse transcriptase inhibitor may be a nucleoside reverse transcriptase inhibitor and/or a nonnucleoside reverse transcriptase inhibitor.

Preferably, a combination according to the invention comprises a quinoline compound as defined above in combination with a strand transfer inhibitor and/or a nucleoside reverse transcriptase inhibitor and/or a nonnucleoside reverse transcriptase inhibitor.

In particular, the strand transfer inhibitor may be selected from the group consisting of L-731,988, L-708,906, L-731,927, L-870,810, L-870,812 and S-1360.

According to preferred embodiments, a combination according to the invention may comprise a quinoline compound as described above in combination with L-731,988 (4-[1-(4-fluorobenzyl)pyrrole-2-yl-]2,4-diketobutanoic acid), and/or a NRTI selected from the group consisting of zidovudine, lamivudine, 2′,3′-dideoxyinosine, and stavudine, and/or a NNRTI which is nevirapine or efavirenz, and/or a protease inhibitor which is indinavir or saquinavir, and/or the entry inhibitor enfuvirtide, and/or the maturation inhibitor PA457.

The combination preferably comprises a quinoline compound in combination with L-731,988, zidovudine, lamivudine, 2′,3′-dideoxyinosine, stavudine, nevirapine, efavirenz, indinavir, saquinavir, or enfuvirtide.

The combination may comprise a quinoline compound in combination with any one of the combinations NNRTI+NRTI marked with a “X” below:

| | zidovudine | lamivudine | 2′,3′-dideoxyinosine | stavudine |
|---|---|---|---|---|
| nevirapine | X | X | X | X |
| efavirenz | X | X | X | X |

The combination may also comprise 8-hydroxy-z-[Z-[(3,4-dihydroxy-5-methoxy-phenyl)ethenyl]]7-quinoline carboxylic acid in combination with L-731,988, and zidovudine, lamivudine, 2′,3′-dideoxyinosine, stavudine, nevirapine, efavirenz, indinavir, saquinavir, or enfuvirtide.

According to a preferred embodiment, a combination according to the invention comprises a quinoline compound as described above in combination with L-731,988, and/or zidovudine, and/or nevirapine.

Additionally, the combination may comprise a quinoline compound in combination with L-731,988, and any one of the combinations NNRTI+NRTI marked with a “X” below:

| | zidovudine | lamivudine | 2′,3′-dideoxyinosine | stavudine |
|---|---|---|---|---|
| nevirapine | X | X | X | X |
| efavirenz | X | X | X | X |

Preferably, the quinoline compound is 8-hydroxy-2-[2-[(3,4-dihydroxy-5-methoxy-phenyl)ethenyl]]7-quinoline carboxylic acid.

The use of a particular combination may be readily decided by the one skilled in the art, for instance in view of emerging viral resistances in the course of HIV infection treatment.

The combination of the present invention has an anti-HIV effect that is greater than the anti-HIV effect of the individual components of the combination when administered alone.

Preferably, the combination of the present invention has therapeutic synergy and provides an improved efficacy over treatment using the components of the combination alone.

A combination manifests therapeutic synergy if it is therapeutically superior to the addition of the therapeutic effects of the independent constituents. The efficacy of a combination may be demonstrated by comparing the IC₅₀ values of the combination with the IC₅₀ values of each of the separate constituents in the study in question. This efficacy may be readily determined by the one skilled in the art. From the IC₅₀ values, a combination index (CI) may be calculated, for instance using the computer program CalcuSyn software from Biosoft, for inhibition efficiencies of 50%, 75% or 90%. The program CalcuSyn performs multiple drug dose-effect calculations using the Median Effect methods described by Chou and Talalay (1983) and Chou and Talalay (1984), which are incorporated herein by reference.

The combination index (CI) equation is based on the multiple dose effect equation of Chou and Talalay derived from enzyme kinetics model. The synergism is defined as a more than expected additive effect and antagonism as a less than expected additive effect. Chou and Talalay proposed the designation of CI=1 as the additive effect. Thus from the multiple drugs effect equation of two drugs, for mutually non exclusive drugs that have totally independent modes of action, CI is calculated as follows:

$$C I = \frac{{( D )}_{1}}{{( Dx )}_{1}} + \frac{{( D )}_{2}}{{( Dx )}_{2}} + \frac{{( D )}_{1} {( D )}_{2}}{{( Dx )}_{1} {( Dx )}_{2}}$$

In the equation, (D)₁ and (D)₂ are the concentrations of drug 1 and 2, respectively, for which x % of inhibition is obeyed in the drug combination. (Dx)₁ and (Dx)₂ are the concentrations of drug 1 and 2 respectively for which x % of inhibition is obeyed for drugs alone.

CI<1, =1 and >1 indicates synergism, additive effect and antagonism respectively.

### Therapeutic Methods

The invention further provides a method of treating a HIV infection, wherein a combination as defined above is administered to a patient in need thereof.

Advantageously, the patient is infected with viruses that show resistance to at least one anti-HIV agent selected from the group consisting of entry (fusion) inhibitors, reverse transcriptase inhibitors, strand transfer inhibitors, protease inhibitors and maturation inhibitors.

The combination may be administered in the form of a single composition comprising the quinoline compound(s) and anti-HIV agent(s) in combination with a pharmaceutically acceptable carrier.

However, the quinoline compound(s) and anti-HIV agent(s) of the combination may be administered each separately, or at least one separately and the other(s) altogether. Each of the quinoline compound(s) and anti-HIV agent(s) may be in the form of a composition, in combination with a pharmaceutically acceptable carrier. Preferably, the quinoline compound and anti-HIV agent(s) are in therapeutically effective amount in the combination.

In the above therapeutic methods, the quinoline compounds and anti-HIV agents may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles.

Such pharmaceutical compositions containing quinoline compounds and anti-HIV agents may thus be in the form of orally-administrable suspensions or tablets; nasal sprays; sterile injectable preparations, for example, as sterile injectable aqueous or oleaginous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters or polyethylene glycols, which are solid at ordinary temperatures, but liquidify and/or dissolve in the rectal cavity to release the drug.

A composition according to the invention may be administered in any of the above routes and according to dosage regimens established in the art. Reference may be made for instance to the “Guidelines for the Use of Antiretroviral Agents in HIV-1-Infected Adults and Adolescents” (Apr. 7, 2005).

The daily dosage of the products may be varied over a wide range from 0.01 to 1,000 mg per adult human per day. For oral administration, the compositions are preferably provided in the form of tablets containing 0.01, 0.05, 0.1, 0.5, 1.0, 2.5, 5.0, 10.0, 15.0, 25.0, 50.0, 100, 250 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. An effective amount of the drug is ordinarily supplied at a dosage level of from about 0.0002 mg/kg to about 20 mg/kg of body weight per day. Preferably, the range is from about 0.001 to 10 mg/kg of body weight per day, and especially from about 0.001 mg/kg to 7 mg/kg of body weight per day. The compounds may be administered on a regimen of 1 to 4 times per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The invention will be further illustrated in view of the following figures and examples.

### FIGURES

FIG. 1 describes the drug efficacy on mutant viruses generated by passaging HIV-1 in the presence of increasing BA011FZ041 concentrations. The integrase-containing PCR fragments of resistant viruses were sequenced. Detected mutations were then introduced into the wild-type NL43 virus by site-directed mutagenesis. Infected cells were treated with BA011FZ401 (black bars) or DKA L-731988 (white bars), and the IC₅₀s were determined. Results are the means of four independent experiments.

FIG. 2 illustrates the activity of BA011FZ041 (first bar from the left in each group) on RTI-resistant HIV-1 strains. AZT (second bar) and 3TC (third bar) were used as nucleic RTIs; efavirenz (fourth bar) and nevirapine (fifth bar) were used as non-nucleoside RTIs.

FIG. 3 illustrates the activity of BA011FZ041 (white bars) and L-731988 (black bars) against genetically engineered DKA-resistant strains.

FIG. 4 shows the isobologrames for BA011FZ041 and nevirapine in ratios 1:1, 1:2, and 2:1.

FIG. 5 shows the isobologrames for BA011FZ041 and zidovudine in ratios 1:1, 1:2, and 2:1.

FIG. 6 shows the isobologrames for BA011FZ041 and L-731,988 in ratios 1:1, 1:2, and 2:1.

### EXAMPLES

### Example 1

### Diketo Acids (DKAs) are Active Against HIV-1 Strains that are Resistant to Quinoline Compounds

Quinoline resistant strains were obtained by passaging the virus in the presence of increasing drug concentrations. The resistant viruses obtained in the presence of 20 μM BA011FZ041 were sequenced, and the identified mutations were introduced into the NL43 wild-type strain by site-directed mutagenesis.

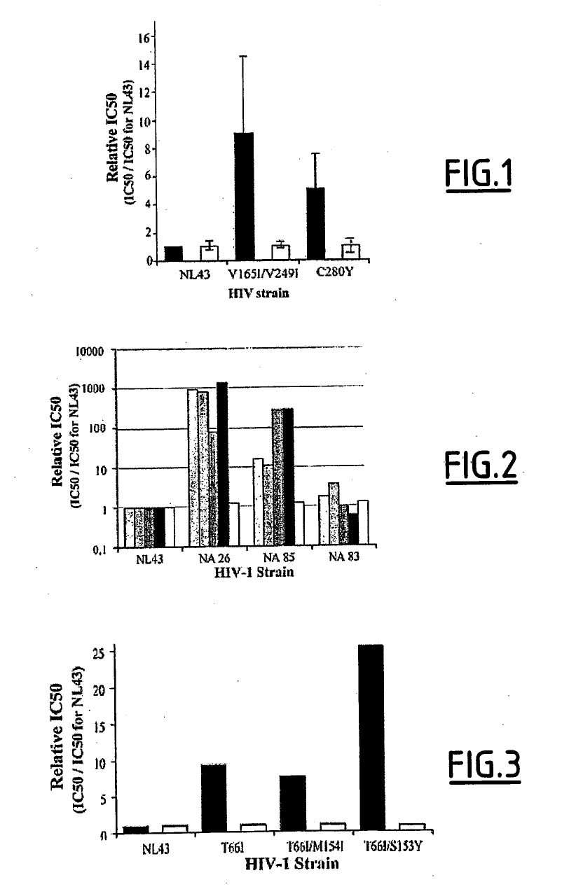

Two mutants appeared after incubation with 20 μM BA011FZ041. One of these mutants contained a single mutation (C280Y), and the other contained a double mutation (V1651 V2491). The IC₅ as with 20 μM BA011FZ041 showed that selected mutations conferred resistance to SQs whereas these viruses remained fully sensitive to DKAs (FIG. 1). The double mutant was the most resistant virus as IC₅₀ BA011FZ041 for it was nearly ninefold higher than that for the wild-type strain. The IC₅₀ of BA011FZ041 for the C280Y mutant was three- to fivefold higher than that for the wild type. In contrast, the IC₅₀ of the DKA integrase inhibitor was not modified for either of these two mutants (FIG. 1).

### Example 2

### Quinoline Compounds are Active Against HIV-1 Strains that Are Resistant to Other Antiviral Compounds

The activity of quinoline compounds against HIV strains that are resistant to reverse transcriptase inhibitors (RTIs) or DKAs was tested.

Three virus strains were used as controls for their resistance to RTIs (Table 4). These viruses harbored mutations in the reverse transcriptase gene, several of which are specifically associated with the resistance to AZT (M41L, D67N, K70N, L210W, R211K, T215F/Y, and K249Q), lamivudine (3TC) (G190A and R211K), nevirapine (K103N, Y181C, and G190A), or efavirenz (K103N and Y181C) or to combinations of RTIs. BA26 and BA85 strains harbor mutations conferring resistance to nucleosidic and non nucleosidic RTIs; BA83's mutations are associated only with resistance to AZT and 3TC.

TABLE 4
Mutations in the reverse transcriptase (RT) gene of the three
RTI-resistant strains.

| NL43 | M41 | V60 | A62 | D67 | T69 | K70 | V75 | F77 | K101 | K103 | F116 | V118 | E122 |
|---|---|---|---|---|---|---|---|---|---|---|---|---|---|
| BA26 | L | | V | N | N | | I | L | Q | | Y | I | K |
| BA85 | | | | N | D | | | | | N | | I | |
| BA83 | | I | | N | N | R | | | | | | I | K |

| NL43 | I135 | Q151 | Y181 | M184 | G190 | Q207 | H208 | L210 | R211 | L214 | T215 | K219 |
|---|---|---|---|---|---|---|---|---|---|---|---|---|
| BA26 | V | M | C | V | A | K/E | Y | | K | | F | Q |
| BA85 | | | | | | | | W | | F | Y | |
| BA83 | T | | | V | | | | | K | | F | Q |

Mutations are indicated with respect to Reverse Transcriptase amino acid numbers.

Three other viruses were mutated in the integrase gene to obtain DKA resistant strains as described previously (Hazuda et al., 2000).

The single-mutant T661 strain, and the double-mutant T661/M1541 and T66I/S153Y strains, were genetically engineered by site directed mutagenesis.

The relative IC₅₀ varied considerably when the drug was an RTI for NA26, BA85, and BA83 strains (FIG. 2) or L-731,988 for integrase mutant strains (FIG. 3). This demonstrates that the mutants were resistant to inhibitors.

In contrast, the relative IC₅₀s for any mutant strains, compared with those for the wild-type NL43 strain, did not vary when BA011FZ041 was used as an inhibitor. This experiment demonstrates that quinoline compounds activity is not impaired by mutations that confer resistance to RTIs or DKAs.

### Example 3

### Quinoline Compounds Show Synergism with DKAs and RTIs

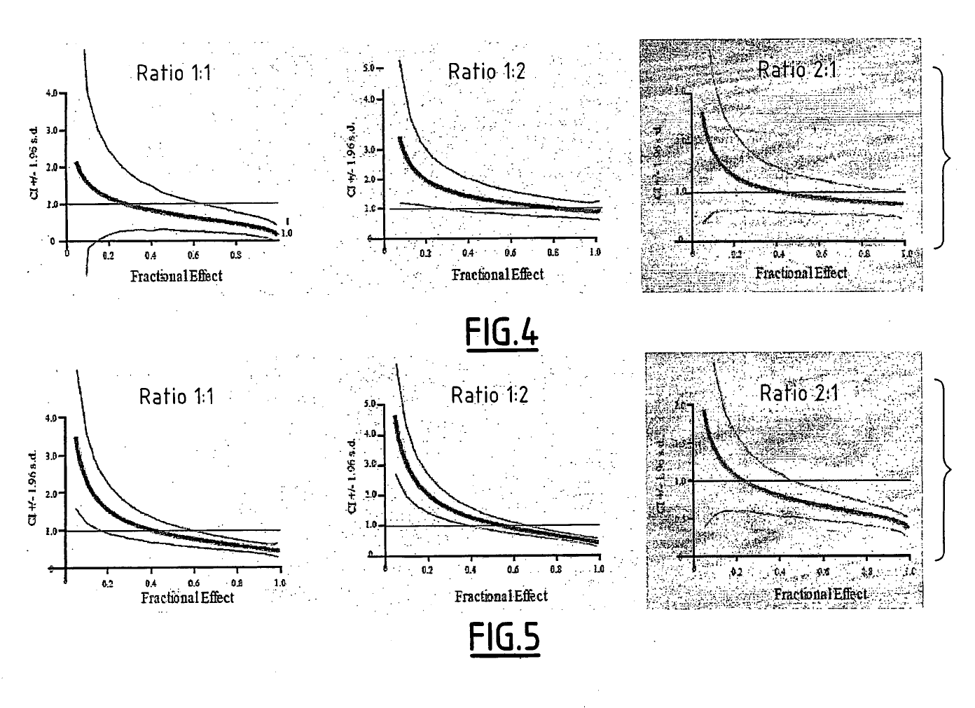

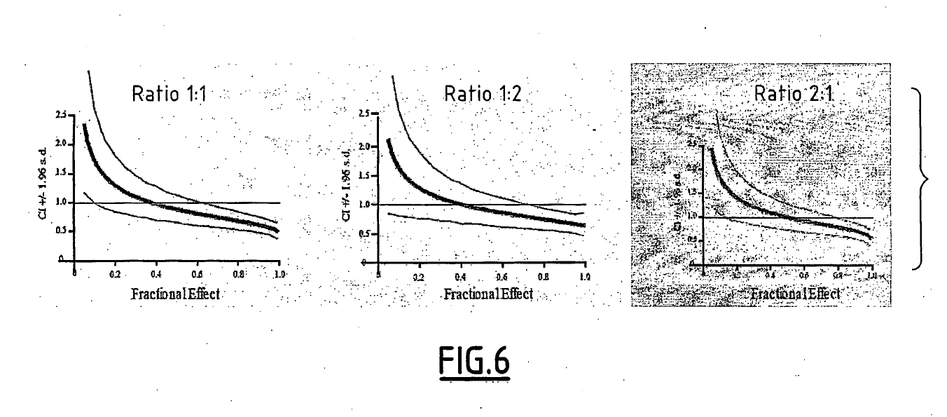

Synergistic interactions between quinoline compounds (BA011FZ041) and either RT (zidovudine and nevirapine) or other integrase inhibitors (L-731,988) were investigated using a NL43 HIV-1 laboratory strain replication assay. Virus infectivity in the presence of inhibitors was monitored with HelaCD4+β-Gal indicator cells (P4 cells). Inhibition by combination of BA011FZ041 and other inhibitors was evaluated at three fixed molar BA011FZ041/Inhibitor ratios: 1:1, 1:2, and 2:1. Three independent experiments with triplicate data points were performed for each ratio. Interactions were calculated by the multiple drug effect equation of Chou and Talalay (Chou and Talalay, 1983; and Chou and Talalay, 1984) based on the median effect principle, using CalcuSyn® software (Biosoft, UK). Efficacy of drug combination was given by the combination index for the effective doses 50, 75 and 90 as described in FIGS. 4, 5 and 6. At a given effective dose, drugs were classically considered synergistic when the upper limit of the 95% confidence interval of combination index was <1 and antagonistic when the lower limit of the 95% confidence was >1.

Material and Methods

### Step 1: Preparation of HeLa P4 Cells in a 96 Flat Bottomed Well Plate

On the day prior to the test, 5,000 HeLaP4 cells per well are seeded in 100 μl of 10% FBS DMEM supplemented with 100 μl/ml penicillin, 100 μg/ml streptomycin and 0.5 mg/ml geniticin (G418). Otherwise, in the morning of the test, 10,000 HeLaP4 cells are seeded per well in 100 μl of 10% FBS DMEM supplemented with 100 μl/ml penicillin, 100 μg/ml streptomycin and 0.5 mg/ml geniticin (G418).

One plate of cells is used for the 2 drugs alone+half a plate per ratio tested is used for development with CPRG and, as necessary, an equivalent number of plates for the MTT toxicity test.

### Step 2: Drug Dilution

The dilutions tested are as follows: 12 IC₅→9 IC₅₀→6 IC₅₀→3 IC₅₀—IC₅₀→IC₅₀/3→IC₅₀/6→IC₅₀/9→IC₅₀/12.

Several drug-drug ratios are tested: 1:1, 1:2, 2:1, 1:4 and 4:1.

Ratios are expressed relative to IC₅₀ values. For the 1:1 ratio, the IC₅₀ concentration for (BA011FZ041+drug X) is the IC₅₀ concentration for BA011FZ041+the IC₅₀ concentration for drug X.

For the 1:2 ratio, the IC₅₀ concentration for (BA011FZ041+drug X) is the IC₅₀ concentration for BA011FZ041+twice the IC₅₀ concentration for drug X.

For the 2:1 ratio, and bearing in mind that the range for BA011FZ041 does not change, the IC₅₀ concentration for (BA011FZ041+drug X) is the IC₅₀ concentration for BA011FZ041+half of the IC₅₀ concentration for drug X.

With, for example, the IC₅₀ for BA011FZ041=5 μM and the IC₅₀ for drug X=10 nM, the IC₅₀ concentration (BA011FZ041+drug X) for the 1:1 ratio is 5 μM BA011FZ041+10 nM drug X.

For the 1:2 ratio, the IC₅₀ concentration (BA011FZ041+drug X) is 5 μM BA011FZ041+20 nM drug X.

For the ratio 2:1, the IC₅₀ concentration (BA011FZ041+drug X) is 5 μM BA011FZ041+5 nM drug X.

The form of BA011FZ041 tested is the sodium carbonate salt.

The test molecules are dissolved in PBS or DMSO:
For PBS: the molecules are resuspended at a concentration 40-fold higher than that finally required in the well so that 5 μl of drug solution are placed in a final volume of 200 μl (100 μl of cells+50 μl of virus suspension+5 or 10 μl of drug solution+45 or 40 μl, of medium). Further dilutions are also performed using PBS and are then distributed at 5 μl per well.
For DMSO: the molecules are resuspended at a concentration 200-fold higher than that finally required in the well so that DMSO be non toxic for the HeLa P4 cells. The quantity of DMSO is constant for each drug concentration tested.

In practice, initial dilutions are performed using DMSO, with these solutions then being rediluted fivefold in PBS or culture medium so as to obtain concentrations 40-fold higher than those finally required in the well. The solutions are then distributed at 5 μl per well.

Prior to infection, the wells are filled up to a final volume of 200 μl with 40 or 45 μl of medium.

### Step 3: Cell Infection

NL4.3 viral supernatant has been produced by CaCl₂ transfection of 293T cells. The HeLa P4 cells are infected with NL 4.3 HIV virus, with about 1 to 2 ng of P24 per well.

Depending on the P24 content of the virus used, a virus solution+10% FBS DMEM supplemented with 100 μl/ml penicillin, 100 μg/ml streptomycin and 0.5 mg/ml geniticin (G418) is prepared and is then distributed at 50 μl per well.

The plates are incubated at 37° C., 5% CO₂, for 60 hours.

The toxicity tests are performed in the absence of infection.

### Step 4: Development with CPRG

The CPRG (chlorophenol red-β-D-galactopyranoside) test is a calorimetric test enabling measurement of the quantity of β-galactosidase produced during viral infection (the β-gal gene is under the control of the TAR element which in turn is controlled by the viral. TAT). The less β-galactosidase there is, the higher the drug's activity.

The supernatant is removed from the plates of HelaP4 cells. 100 μl of CPRG lysis buffer is added per well (for 110 wells: Na₂HPO₄ (0.5M) 1687 μL, NaH₂PO₄ (1M) 440 μL, KCl (1M) 110 μL, MgSO₄ (1M) 220 μL, EDTA (0.5M) 55 μL, β-mercaptoethanol 39 μL, NP40 14 μL, water 8617 μL).

The cells are incubated for 10 minutes at 37° C. in order to obtain full cell lysis. The lysed wells are homogenized and 50 μl of each lysate is transferred into the corresponding well of another plate containing 100 μl reaction buffer (for 110 wells: pH 7.4 phosphate buffer (1M) 880 μL, MgCl₂ 99 μL, β-mercaptoethanol 77 μL, water 8140 μL, CPRG (250 mg of CPRG dissolved in 9.1 ml of Millipore water+455 μl pH 7.4 sodium phosphate buffer) 1833 μL).

The color is let to develop at 37° C. for between 30 min and 2 h.

The OD is read at 560 nm with a 690 nm reference filter.

### Step 5: MTT (3-(4,5-dimethylthiazol-2-yl)-2,5-diphenyltetrazolium bromide) Toxicity Test

The test conditions have been defined so that the virus concentration used does not itself have a cytotoxic effect. Hence, the toxicity tests are performed in the absence of infection. If toxicity is detected, it can only be due to the drug(s).

100 μl of medium are removed per well. 10 μl of a 5 mg/ml MTT solution are added to each well. Incubation is carried out at 37° C. for 24 h. The MTT forms deep violet formazan crystals, a sign of mitochondrial activity.

100 μl of MTT lysis buffer (10 g of sodium dodecyl sulfate (SDS) dissolved in 50 ml of 1:1 water-dimethylformamide (DMF) mixture+110 μl of 1N HCl+110 μl 80% acetic acid, pH 4.7) are added and the wells are incubated at 37° C. for 24 h. The wells are homogenized and 100 μl of lysate are transferred into a clean 96 flat bottomed well plate. The OD is read at 560 nm with a 690 nm reference filter.

### Step 6: Analysis of the Results

The CPRG results are analyzed using CalcuSyn software from Biosoft. This computer program enables definition of the combination index (CI) for inhibition efficiencies of 50%, 75% or 90%.

If the CI is less than 1, the effect is synergistic. If the CI is equal to 1, the effect is additive. If the CI is greater than 1, the effect is antagonistic.

Furthermore, this program enables definition (at the IC₅₀ of the drug combination) of the concentration of each drug in the mixture and comparison of this concentration with the IC₅₀ of the drug alone.

Results

Synergy was considered significant when the 95% confidence interval of the CI from three independent experiments was <1.

TABLE 5
Combination index for BA011FZ041 and Nevirapine

| | Combination index (95% IC) for % inhibition of | | | | | | | | |
|---|---|---|---|---|---|---|---|---|---|
| | Ratio 1:1 | | | Ratio 1:2 | | | Ratio 2:1 | | |
| | 50 | 75 | 90 | 50 | 75 | 90 | 50 | 75 | 90 |
| Mean | 0.967 | 0.833 | 0.758 | 1.079 | 0.909 | 0.819 | 0.964 | 0.884 | 0.837 |
| Higher | 1.097 | 0.931 | 0.851 | 1.100 | 0.973 | 0.880 | 1.042 | 0.984 | 0.946 |
| value | | | | | | | | | |
| Lowest | 0.820 | 0.752 | 0.710 | 1.024 | 0.832 | 0.724 | 0.838 | 0.771 | 0.746 |
| value | | | | | | | | | |

TABLE 6
IC₅₀ values for BA011FZ041 and Nevirapine

| | IC₅₀ values for drugs alone and in the mix | | | | | | | | | | | |
|---|---|---|---|---|---|---|---|---|---|---|---|---|
| | Ratio 1:1 | | | | Ratio 1:2 | | | | Ratio 2:1 | | | |
| | FZ41 | | Nev | | FZ4 | | Nev | | FZ41 | | Nev | |
| | (μM) | | (nM) | | (μM) | | (nM) | | (μM) | | (nM) | |
| Mean | 1.6 | 1.1 | 39 | 11 | 1.5 | 0.9 | 41 | 18 | 1.5 | 1.2 | 39 | 6.3 |
| Higher | 1.7 | 1.3 | 41 | 13 | 1.73 | 1.03 | 43 | 20 | 1.6 | 1.4 | 41 | 7 |
| value | | | | | | | | | | | | |
| Lowest | 1.4 | 0.9 | 38 | 09 | 1.26 | 0.82 | 40 | 16 | 1.4 | 1.16 | 38 | 5.8 |
| value | | | | | | | | | | | | |

TABLE 7
Combination index for BA011FZ041 and Zidovudine (ZVD)

| | Combination index (95% IC) for % inhibition of | | | | | | | | |
|---|---|---|---|---|---|---|---|---|---|
| | Ratio 1:1 | | | Ratio 1:2 | | | Ratio 2:1 | | |
| | 50 | 75 | 90 | 50 | 75 | 90 | 50 | 75 | 90 |
| Mean | 1.15 | 0.79 | 0.60 | 1.069 | 0.818 | 0.723 | 0.93 | 0.786 | 0.722 |
| Higher | 1.457 | 1.000 | 0.731 | 1.253 | 0.919 | 0.964 | 1.076 | 0.889 | 0.864 |
| value | | | | | | | | | |
| Lowest | 0.786 | 0.583 | 0.492 | 0.973 | 0.658 | 0.509 | 0.741 | 0.601 | 0.506 |
| value | | | | | | | | | |

TABLE 8
IC₅₀ values for BA011FZ041 and Zidovudine (ZVD)

| | IC₅₀ values for drugs alone and in the mix | | | | | | | | | | | |
|---|---|---|---|---|---|---|---|---|---|---|---|---|
| | Ratio 1:1 | | | | Ratio 1:2 | | | | Ratio 2:1 | | | |
| | FZ41 (μM) | | ZVD (nM) | | FZ41 (μM) | | ZVD (nM) | | FZ41 (μM) | | ZVD (nM) | |
| Mean | 1.4 | 0.8 | 29 | 16 | 1.7 | 0.7 | 46 | 28 | 1.7 | 1.1 | 44 | 11 |
| Higher | 1.7 | 0.9 | 30 | 18 | 2 | 0.95 | 74 | 38 | 2 | 1.18 | 69 | 11.8 |
| value | | | | | | | | | | | | |
| Lowest | 1.4 | 0.9 | 28 | 12 | 1.5 | 0.48 | 28 | 19.5 | 1.5 | 0.48 | 27.8 | 10 |
| value | | | | | | | | | | | | |

TABLE 9
Combination index for BA011FZ041 and L-731,988 DKA

| | Combination index (95% IC) for % inhibition of | | | | | | | | |
|---|---|---|---|---|---|---|---|---|---|
| | Ratio 1:1 | | | Ratio 1:2 | | | Ratio 2:1 | | |
| | 50 | 75 | 90 | 50 | 75 | 90 | 50 | 75 | 90 |
| Mean | 1.00 | 0.844 | 0.756 | 1.00 | 0.825 | 0.749 | 1.00 | 0.922 | 0.888 |
| Higher | 1.213 | 0.972 | 0.874 | 1.132 | 1.039 | 0.985 | 1.093 | 0.985 | 0.998 |
| value | | | | | | | | | |
| Lowest | 0.786 | 0.653 | 0.578 | 0.775 | 0.672 | 0.624 | 0.899 | 0.776 | 0.687 |
| value | | | | | | | | | |

TABLE 10
IC₅₀ values for BA011FZ041 and L-731,988 DKA

| | IC₅₀ values for drugs alone and in the mix | | | | | | | | | | | |
|---|---|---|---|---|---|---|---|---|---|---|---|---|
| | Ratio 1:1 | | | | Ratio 1:2 | | | | Ratio 2:1 | | | |
| | | | L-731,988 | | | | L-731,988 | | | | L-731,988 | |
| | FZ41 (μM) | | (μM) | | FZ41 (μM) | | (μM) | | FZ41 (μM) | | (μM) | |
| Mean | 1.3 | 0.9 | 1.5 | 0.3 | 1.3 | 0.8 | 1.5 | 0.5 | 1.3 | 1.1 | 1.5 | 0.2 |
| Higher | 1.7 | 1.3 | 2.5 | 0.43 | 1.74 | 1.18 | 2.3 | 0.73 | 1.7 | 1.6 | 2.5 | 0.23 |
| value | | | | | | | | | | | | |
| Lowest | 1.0 | 0.8 | 0.96 | 0.26 | 1.0 | 0.57 | 1.0 | 0.46 | 1.0 | 0.7 | 1.0 | 0.15 |
| value | | | | | | | | | | | | |

Combination of BA011FZ041 with nevirapine led to a synergistic effect at effective dose (ED)75 and ED90 for all three ratios.

Combination of BA011FZ041 with zidovudine demonstrated synergy at ED90 for all three ratios and a synergic effect at the ED75 for ratios 1:2 and 2:1.

For nevirapine and zidovudine, IC₅₀ of these drugs in combination with BA011FZ041 were decreased by a factor 2 to 6 as compared to IC₅₀ for the drugs alone. Furthermore, combination of the two integrase inhibitors led also to synergistic effect at ED75 and ED90 for the ratios 1:1 and 2:1. For the ratio 1:2, a synergic effect was found at ED90 although a mere additive effect was detected at ED75. Finally, IC₅₀ of L-731,988 was significantly decreased by a factor 7 when it was present in combination with quinoline compounds, thus emphasising the complementarities of both classes of anti-integrase agents.

### REFERENCES

Di Santo R, Costi R. Artico M, Tramontano E, La Colla P, and Pani A (2003) HIV-1 integrase inhibitors that block HIV-1 replication in infected cells. Planning synthetic derivatives from natural products. Pure Appl. Chem., Vol. 75, Nos. 2-3, pp. 195-206.
Brown P O (1997) Integration, in Retroviruses (Coffin J M, Hughes S H, and Varmus H E, eds) pp 161-203, Cold Spring Harbor Laboratory, Cold Spring Harbor, N.Y.
Hazuda D J, Felock P, Witmer M, Wolfe A, Stillmock K, Grobler J A, Espeseth A, Gabryelski L, Schleif W, Blau C, et al. (2000) Inhibitors of strand transfer that prevent integration and inhibit HIV-1 replication in cells. Science (Wash D.C.) 287:646-650.
Hazuda D J, Anthony N J, Gomez R P, Jolly S M, Wai J S, Zhuang L, Fisher T E, Embrey M, Guare J P Jr, Egbertson M S, Vacca J P, Huff J R, Felock P J, Witmer M V, Stillmock K A, Danovich R, Grobler J, Miller M D, Espeseth A S, Jin L, Chen I W, Lin J H, Kassahun K, Ellis J D, Wong B K, Xu W, Pearson P G, Schleif W A, Cortese R, Emini E, Summa V, Holloway M K, Young S D. (2004) A naphthyridine carboxamide provides evidence for discordant resistance between mechanistically identical inhibitors of HIV-1 integrase. Proc Natl Acad Sci USA. 101 (31):11233-8.
Chou T C, Talalay P. (1984) Quantitative analysis of dose-effect relationships: the combined effects of multiple drugs or enzyme inhibitors. Adv Enzyme Regul. 22:27-55.
Chou T. C., Talalay P. (1983) Analysis of combined drug effects: a new look at a very old problem. Trends Pharmacol. Sci., 4: 450-454.

1. A combination having therapeutic synergy comprising a compound of formula (I) 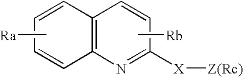
in which Ra, Rb and Rc, identical or different from one another, represent one or more substituents, themselves identical or different, occupying any position on the rings, the substituents being chosen from a —(CH₂)n-Y or —CH—CH—Y group, where Y represents a halogen atom, an —OH, —OR, —COH, —COR, —COOH, —COOR, —COH, —COR, —CONH₂, —CON(Rx,Ry)1-CH—NOH, —CO—CH—NOH, —NRxRy, —NO₂, —PO(OR)₂, —SH₂, —SH, —SR, SO₂R, —SO₂NHR, —CN, —NH(C═O)R, or Z(Rc) radical, where
R represents an alkyl radical with 1 to 8 carbon atoms, or an aryl or heterocyclic radical, Rx and Ry, identical or different, represent an hydrogen atom or a linear or branched alkyl radical with 1 to 5 carbon atoms,
Z represents an aryl, heterocyclic radical or an aromatic ring containing heteroatoms chosen from O, N or S, as substitutions for the carbon atoms constituting said aromatic ring, it being possible or otherwise for this ring to be substituted with R^{c} and
n is zero or an integer from 1 to 5,
Rb moreover can represent a hydrogen atom, and when Y represents a —COOH or —COOR group in Rc, Z, if it represents an aryl group, includes at least 3 substituents or the quinoline ring is trisubstituted,
X represents an ethylene double bond, or a —(CH₂)n- group, where n is an integer from 1 to 5, or a —(C═O)N(Rd)X′— group, or a —CH(Rd)—CH(Re)— group, Rd and Re, identical or different, representing a hydrogen atom, halogen atom, a hydroxy or epoxy group, or a —(CH₂)n′-O—C(O)—(CH₂)m-, —(CH₂)n′-C(O)—O—(CH₂)m-, —(CH₂)IV—O— (CH₂)ITi—, —(CH₂)n′-N(O)—(CH₂)m- or —(CH₂)n′-S(O)t-(CH₂)m- group, where n′ is an integer from 0 to 8,
Rd represents a hydrogen atom or a group —(CH₂)n′-Y\ for which n″ is equal to 0, 1, 2 or 3 and Y′ represents —CH₃, —COOH, —COOR′, —CN, —OH, —OR′, SR′, or an aryl group optionally substituted with Rc, R′ represents a linear or branched alkyl chain of 1 to 4 carbon atoms,
X′ represents an alkyl-(CH₂)n′″— chain in which n′″ is equal to 0, 1 or 2, or O, or N,
m is an integer from 0 to 8, t is zero or an integer equal to 1 or 2, and Q represents a hydrogen atom, an alkyl or aryl radical, as well as the pharmaceutically acceptable salts of these compounds, their diastereoisomeric forms and their enantiomeric forms,
in combination with at least one HIV infection therapeutic agent selected from the group consisting of an entry inhibitor, a reverse transcriptase inhibitor, a strand transfer inhibitor, a protease inhibitor, and a maturation inhibitor.

2. The combination according to claim 1, wherein said compound of formula (I) has formula (Ia): 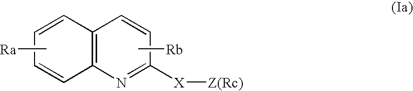
in which Ra, Rb and Rc, identical or different from one another, represent one or more substituents, themselves identical or different, occupying any position on the rings, the substituents being chosen from a —(CH₂)n-Y or —CH—CH—Y group, where Y represents a halogen atom, an —OH, —OR, —COH, —COR, —COOH, —COOR, —COH, —COR, —CONH₂, —CON(Rx,Ry), —CH—NOH, —CO—CH—NOH, —NH₂, —N(Rx,Ry), —NO₂, —PO(OR)₂, —SH₂, —SR, —SO₂R, —SO₂NHR, —NH(C═O)R, —CN, or Z(Rc) radical, where R represents an alkyl radical with 1 to 8 carbon atoms, or an aryl or heterocyclic radical, Rx and Ry, identical or different, represent an alkyl radical with 1 to 5 carbon atoms, Z represents an aryl or heterocyclic radical and n is zero or an integer from 1 to 5, Rb moreover can represent a hydrogen atom, and when Y represents a —COOH or —COOR group in Rc, Z, if it represents an aryl group, includes at least 3 substituents or the quinoline ring is trisubstituted, X represents an ethylene double bond, or a —(CH₂)n- group, where n is an integer from 1 to 5, or a —CH(Rd)—CH(Re)— group, R^{d} and Re, identical or different, representing a hydrogen atom, halogen atom, a hydroxy or epoxy group, or a —(CH₂)n′-O—C(O)—(CH₂)m-, —(CH₂)n′-C(O)—O—(CH₂)m-, —(CH₂)n′-O—(CH2)m-, —(CH₂)n′-N(Q)-(CH₂)m- or —(CH₂)n′-S(O)t-(CH₂)m- group, where n′ is an integer from 0 to 8, m is an integer from 0 to 8, t is zero or an integer equal to 1 or 2, and Q represents a hydrogen atom, an alkyl or aryl radical,
as well as the pharmaceutically acceptable salts of these derivatives, their diastereoisomeric forms and their enantiomeric forms.

3. The combination according to claim 2, wherein said compound of formula (Ia) is selected from the group consisting of:
8-hydroxy-2-[2-[(3,4-dihydroxy-5-methoxy-phenyl)ethenyl]]5,7-quinoline dicarboxylic acid
8-hydroxy-2-[2-[(3,4-dihydroxy-phenyl)ethenyl]]5,7-quinoline dicarboxylic acid
8-hydroxy-2-[2-[(3,4-dihydroxy-phenyl)ethenyl]]5-quinoline carboxylic acid
2-[2-[(3,4-dihydroxy-phenyl)ethenyl]]-quinoline
8-hydroxy-2-[2-[(3,4-dihydroxy-phenyl)ethenyl]]-quinoline
7-cyano-8-hydroxy-2-[2-[(3-acetamido-4-hydroxy-5-methoxy-phenyl)ethenyl]]-quinoline
2-[2-[(3,4-dihydroxy-phenyl)ethenyl]]7,8-quinoline dicarboxylic acid
8-hydroxy-2-[2-[(3,4-dihydroxy-5-methoxy-phenyl)ethenyl]]7-quinoline carboxylic acid
8-hydroxy-2-[2-[(3-methoxy-4-hydroxy-phenyl)ethenyl]]7-quinoline carboxylic acid
8-hydroxy-2-[2-[(2,3-dihydroxy-phenyl)ethenyl]]7-quinoline carboxylic acid
8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3,5-dimethoxy-4-hydroxy-phenyl)ethenyl]]7-quinoline carboxylic acid 8-hydroxy-2-[2-[(3-amino-4-hydroxy-5-methoxy-phenyl)ethenyl]]7-quinoline carboxylic acid
8-hydroxy-2-[2-[(3,5-dibromo-4-hydroxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid
8-hydroxy-2-[2-[(3,4-dihydroxy-phenyl)ethenyl]]7-quinoline carboxylic acid
8-hydroxy-2-[2-[(3,4,5-trihydroxy-phenyl)ethenyl]]7-quinoline carboxylic acid
as well as their pharmaceutically acceptable salts, their diastereoisomeric forms and their enantiomeric forms

4. The combination according to claim 2, wherein said compound of formula (Ia) has formula (Ia′): 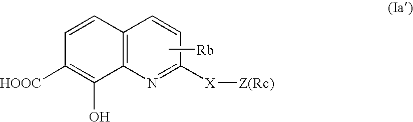
wherein Rb, X, Z, Rc are defined as in formula (Ia).

5. The combination according to claim 4, wherein said compound of formula (Ia′) is selected from the group consisting of:
8-hydroxy-2-[2-[(3,4-dihydroxy-5-methoxy-phenyl)ethenyl]]7-quinoline carboxylic acid
8-hydroxy-2-[2-[(3-methoxy-4-hydroxy-phenyl)ethenyl]]7-quinoline carboxylic acid
8-hydroxy-2-[2-[(2,3-dihydroxy-phenyl)ethenyl]]7-quinoline carboxylic acid
8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3,5-dimethoxy-4-hydroxy-phenyl)ethenyl]]7-quinoline carboxylic acid
8-hydroxy-2-[2-[(3-amino-4-hydroxy-5-methoxy-phenyl)ethenyl]]7-quinoline carboxylic acid
8-hydroxy-2-[2-[(3,5-dibromo-4-hydroxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid
8-hydroxy-2-[2-[(3,4-dihydroxy-phenyl)ethenyl]]7-quinoline carboxylic acid
8-hydroxy-2-[2-[(3,4,5-trihydroxy-phenyl)ethenyl]]7-quinoline carboxylic acid
as well as their pharmaceutically acceptable salts, their diastereoisomeric forms and their enantiomeric forms.

6. The combination according to claim 1, wherein said compound of formula (I) has formula (Ib): 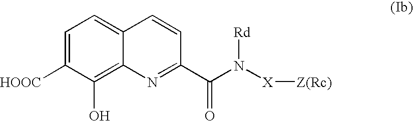
in which
X represents an alkyl-(CH₂)n- chain in which n is equal to 0, 1 or 2, or O or N,
Z represents an aromatic ring which may contain heteroatoms chosen from O, N or S, as substitutions for the carbon atoms constituting said aromatic ring, it being possible or otherwise for this ring to be substituted with Rc,
Rc represents 1 to 3 identical or different substituents chosen from the groups —OH, —OR, —COOH, —COOR, —COH, —COR, —NH₂, —NH(R), —NH(R′R′), —SH, —SR and CN,
Rd represents a hydrogen atom or a group —(CH₂)n″-Y′ for which n″ is equal to 0, 1, 2 or 3 and Y′ represents —CH₃, —COOH, —COOR′, —CN, —OH, —OR′, SR′, or an aryl group optionally substituted with Rc,
R and R′, which are identical or different, represent a linear or branched alkyl chain of 1 to 4 carbon atoms, and their pharmaceutically acceptable salts, their diastereoisomeric forms and their enantiomeric forms.

7. The combination according to claim 6, wherein said compound of formula (Ib) is selected from the group consisting of:
2-(2,3,4-trihydroxy-benzylcarbamoyl)-8-hydroxyquinoline-7-carboxylic acid
2,4-dihydroxy-b[theta]nzylcarbamoyl)-8-hydroxyquinoline-7-carboxylic acid
2-(3,4-dihydroxy-5-methoxy-phenylcarbamoyl)-8-hydroxyquinoline-7-carboxylic acid
2-(3,4-dihydroxy-benzylcarbamoyl)-8-hydroxyquinoline-7-carboxylic acid
2-(2,3-dihydroxy-benzylcarbamoyl)-8-hydroxyquinoline-7-carboxylic acid
2-(3,4-dihydroxy-phenylcarbamoyl)-8-hydroxyquinoline-7-carboxylic acid
as well as the pharmaceutically acceptable salts of these derivatives, their diastereoisomeric forms and their enantiomeric forms.

8. The combination according to claim 1, wherein the at least one HIV infection therapeutic agent is a reverse transcriptase inhibitor.

9. The combination according to claim 8, which comprises at least one nucleoside reverse transcriptase inhibitor and at least one nonnucleoside reverse transcriptase inhibitor.

10. The combination according to claim 8, wherein said reverse transcriptase inhibitor is a nucleoside reverse transcriptase inhibitor selected from the group consisting of 3TC, AZT (3′-azido-3′-deoxythymidine), azidothymidine, abacavir, d4T, didanosine, 2′,3′-dideoxyinosine (ddI), 2′,3′-dideoxycytidine (ddC), emtricitabine, FTC, lamivudine, stavudine, tenofovir disoproxil/emtricitabine, tenofovir disoproxil fumarate, zalcitabine, zidovudine, alovudine ((3′-Fluoro-3′-deoxythymidine), amdoxovir (2R-cis-4-(2,6-diamino-9H-purin-9-yl)-1,3-dioxolane-2-methanol), and elvucitabine (2,3-Dideoxy-2,3-didehydro-beta-L-fluorocytidine).

11. The combination according to claim 8, wherein said reverse transcriptase inhibitor is a nonnucleoside reverse transcriptase inhibitor selected from the group consisting of delavirdine, efavirenz, nevirapine, calanolide A (2H,6H,10H-Benzo(1,2-b:3,4-b′:5,6-b″)tripyran-2-one, 11,12-dihydro-12-hydroxy-6,6, 10,11-tetramethyl-4-propyl-, (10R-(1 Oalpha, 11 beta, 12alpha)), capravirine (5-(3,5-dichlorophenyl)thio-4-isopropyl-1-(4-pyridyl)methyl-1H-imidazol-2-ylmethyl carbamate), etravirine (4-[[6-amino-5-bromo-2-[(4-cyanophenyl)amino]-4-pyrimidinyl]oxy]-3,5-dimethylbenzonitrile), TMC-120 (4-({4-[(2,4,6-Trimethylphenyl)amino]pyrimidin-2-yl}amino)benzenecarbonitrile), TMC-278 ((E) 4-[[4-[[4-(2-cyanoethenyl)-2,6-dimethylphenyl]amino]-2-pyrimidinyl]amino]benzonitrile), and BMS-561390 (2(1H)-Quinazolinone, 6-chloro-4-[(1E)-2-cyclopropylethenyl]-3,4-dihydro-4-(trifluoromethyl)-, (4S)1-).

12. The combination according to claim 1, wherein the at least one HIV infection therapeutic agent is a strand transfer inhibitor.

13. The combination according to claim 12, wherein said strand transfer inhibitor is selected from the group consisting of L-731,988 (4-[1-(4-fluorobenzyl)pyrrole-2-yl-]2,4-diketobutanoic acid), L-708,906 (4-(3,5-Bis-benzyloxy-phenyl)-2,4-dioxo-butyric acid), L-731,927 (1H-Pyrrole-2-butanoic acid, a,g-dioxo-1-(3-phenylpropyl)-), L-870,810 (5-(1,1-dioxido-1,2-thiazinan-2-yl)˜N-(4-fluorobenzyl)-8-hydroxy-1,6-naphthyridine-7-carboxamide), L-870,812 (8-hydroxy-5-N-methyl-N′-(2-dimethylamino-1,2-diketo)ethylamino-1,6-naphthyridine-7-(4′-fluorobenzyl)-carboxamide) and S-1360 (1-[5-(4-fluorobenzyl)furan-2-yl]-3-hydroxy-3-(1H-1,2,4-triazol-3-yl)-propenone).

14. The combination according to claim 12, wherein said strand transfer inhibitor is L-731,988 (4-[1-(4-fluorobenzyl)pyrrole-2-yl]-2,4-diketobutanoic acid).

15. The combination according to claim 1, wherein the at least one HIV infection therapeutic agent is a protease inhibitor.

16. The combination according to claim 15, wherein said protease inhibitor is selected from the group consisting of amprenavir, atazanavir, fosamprenavir, indinavir, lopinavir, mesylate, nelfinavir, ritonavir, saquinavir, tipranavir (2-Pyridinesulfonamide, N-[3-[(1R)-1-[(6R)-5,6-dihydro-4-hydroxy-2-oxo-6-(2-phenylethyl)-6-propyl-2H-pyran-3-yl]propyl]phenyl]-5-(trifluoromethyl)-) and TMC-114 CAS Number: 618109-00-5).

17. The combination according to claim 1, wherein the at least one HIV infection therapeutic agent is an entry inhibitor.

18. The combination according to claim 17, wherein said entry inhibitor is selected from the group consisting of enfuvirtide, PRO-542 (CAS Registry Number 383198-58-1), TNX-355, SCH-417690 ((1-(4,6-Dimethyl-pyrimidin-5-yl)-1-(4-{(S)-4-[(R)-2-methoxy-1-(4-trifluoromethyl-phenyl)-ethyl]-3-methyl-piperazin-1-yl}-4-methyl-piperidin-1-yl)-methanone), GSK-873,140 (Benzoic acid,4-(4-(((3R)-1-butyl-3-((R)-cyclohexylhydroxymethyl)-2,5-dioxo-1,4,9-triazaspiro(55)undec-9-yl)methyl)phenoxy)), and maraviroc (CAS number 376348-65-1).

19. The combination according to claim 1, wherein the at least one HIV infection therapeutic agent is a maturation inhibitor.

20. The combination according to claim 19, wherein said maturation inhibitor is PA-457 (3-O-(3′,3′-dimethylsuccinyl) betulinic acid).

21. The combination according to claim 1, which comprises L-731,988 (4-[1-(4-fluorobenzyl)pyrrole-2-yl-]2,4-diketobutanoic acid), and/or a nucleoside reverse transcriptase inhibitor selected from the group consisting of zidovudine, lamivudine, 2′,3′-dideoxyinosine, and stavudine, and/or a nonnucleoside reverse transcriptase inhibitor which is nevirapine or efavirenz, and/or a protease inhibitor which is indinavir or saquinavir, and/or the entry inhibitor enfuvirtide, and/or the maturation inhibitor PA-457.

22. The combination according to claim 21, which comprises L-731,988 (4-[1-(4-fluorobenzyl)pyrrole-2-yl-]2,4-diketobutanoic acid), and/or zidovudine, and/or nevirapine.

23. The combination according to claim 21, wherein said compound of formula (I) is 8-hydroxy-2-[2-[(3,4-dihydroxy-5-methoxy-phenyl)ethenyl]]7-quinoline carboxylic acid.

24. A method of treating a HIV infection, wherein a combination according to claim 1 is administered to a patient in need thereof.

25. A compound of formula (I) selected from the group consisting of:
8-hydroxy-2-[2-[(3,4-dihydroxy-5-methoxy-phenyl)ethenyl]]5,7-quinoline dicarboxylic acid
8-hydroxy-2-[2-[(2,3-dihydroxy-phenyl)ethenyl]]7-quinoline carboxylic acid
8-hydroxy-2-[2-[(3,4-dihydroxy-phenyl)ethenyl]]5-quinoline carboxylic acid 8-hydroxy-2-[2-[(3-methoxy-4-hydroxy-5-iodo-phenyl)ethenyl]]7-quinoline carboxylic acid
8-hydroxy-2-[2-[(3-nitro-4-hydroxy-5-methoxy-phenyl)ethenyl]]7-quinoline carboxylic acid
8-hydroxy-2-[2-[(3-amino-4-hydroxy-5-methoxy-phenyl)ethenyl]]7-quinoline carboxylic acid
8-hydroxy-2-[2-[(3-acetamido-4-hydroxy-5-methoxy-phenyl)ethenyl]]7-quinoline carboxylic acid
8-hydroxy-2-[2-[(4-acetamido-phenyl)ethenyl]]7-quinoline carboxylic acid as well as the pharmaceutically acceptable salts of these derivatives, their diastereoisomeric forms and their enantiomeric forms.

26. A composition comprising a compound according to claim 25 in a pharmaceutically acceptable carrier.

27. A method of treating a HIV infection comprising administering a patient in need thereof with a composition according to claim 26.
